(19) United States
(12) Patent Application Publication
(10) Pub. No.: US 2025/0090599 A1
(43) Pub. Date: Mar. 20, 2025

(54) METHODS AND COMPOSITIONS FOR TREATING INFLAMMATORY SKIN DISEASE WITH RECOMBINANT MICROORGANISMS

(71) Applicant: AZITRA INC., FARMINGTON, CT (US)

(72) Inventor: Travis Michael Whitfill, Dallas, TX (US)

(21) Appl. No.: 18/641,696

(22) Filed: Apr. 22, 2024

Related U.S. Application Data
(63) Continuation of application No. 16/121,903, filed on Sep. 5, 2018, now Pat. No. 12,208,124.
(60) Provisional application No. 62/685,687, filed on Jun. 15, 2018.
(60) Provisional application No. 62/554,271, filed on Sep. 5, 2017.

(51) Int. Cl. A61K 35/74; A61K 38/17; A61K 39/085; A61P 17/00; C07K 14/71

(52) U.S. Cl. CPC A61K 35/74; A61K 38/179; A61K 39/085; A61P 17/00; C07K 14/71; C07K 2319/10

(57) ABSTRACT

The present invention provides isolated plasmids, recombinant microorganisms, kits, and methods for the treatment of inflammatory skin disease.

### RELATED APPLICATIONS

This application is a continuation of U.S. patent application Ser. No. 16/121,903, filed Sep. 5, 2018, which claims priority to U.S. Provisional Application 62/554,271 filed on Sep. 5, 2017, and U.S. Provisional Application 62/685,687 filed on Jun. 15, 2018, the entire contents of both of which are incorporated by reference in their entireties herein.

### SEQUENCE LISTING

The instant application contains a Sequence Listing which has been submitted electronically in XML file format and is hereby incorporated by reference in its entirety. Said XML copy, created on Jun. 20, 2024, is named 129062-00104_SL.xml and is 119,230 bytes in size.

### BACKGROUND OF THE INVENTION

Atopic dermatitis (AD), or eczema, is a chronic, pruritic, inflammatory skin disease that affects 5-20% of children worldwide (Williams, H., et al. J Allergy Clin. Immunol. 1999;103(1 Pt 1):125-138), and is also prevalent in many adults. The prevalence of atopic dermatitis is increasing, with some form of atopic dermatitis affecting 11% of the U.S. population (Shaw, T. E., et al. J Invest Dermatol. 2011;131(1):67-73), or about 35 million people, with direct costs of $5 billion in the U.S. alone. The primary features of the disease are dry, scaly, itchy skin. Despite the increasing prevalence of atopic dermatitis worldwide and its significant disease burden, few targeted and effective treatment options are available. Notably, the most commonly used treatment methods include broad, non-specific approaches, including, but not limited to, skin hydration, bleach baths, UV treatment, dietary interventions, antimicrobials, antihistamines, systemic immunomodulatory agents, and the administration of topical corticosteroids (Hoare, C., et al. Health Technol. Assess. 2000;4(37):1-191). However, despite these numerous options, few provide long-lasting resolution of symptoms, and atopic dermatitis recurrence is common in most individuals. Further, a 2013 National Health and Wellness Survey revealed a significant associated burden on atopic dermatitis patients, who, when compared with non-atopic dermatitis patients, reported higher levels of healthcare resources (healthcare provider/ER visits), lower health-related quality of life, and nearly twice as much lost work productivity. Moreover, atopic dermatitis patients had markedly higher prevalence of allergies (46% vs. 20%), asthma (22% vs. 8%), anxiety (43% vs. 21%), and depression (37% vs. 21%) (Whiteley, J., et al. Current Medical Research and Opinion. 2016:1-32). Accordingly, there is a large unmet need in view of the significant burden atopic dermatitis has on our healthcare system.

Recent research has elucidated the pathophysiology of atopic dermatitis and has revealed that a skin barrier defect is in many cases primarily responsible for the onset of atopic dermatitis, which results in both transepidermal water loss (TEWL or TWL) and increased antigen and pathogen exposure. Concurrently, atopic dermatitis is often characterized by dysbiosis (or a microbial imbalance, the severity of which is associated with disease severity; see Kong, H. H., et al. Genome research. 2012;22(5):850-859; and FIG. 1), which is a notable feature of atopic dermatitis, and a lack of diversity of the skin microbiome, which is dominated by Staphylococcus aureus during atopic dermatitis flares and untreated skin. Finally, there is an activated inflammatory response, particularly driven by, inter alia, IL-4/IL-13, with a predominate T-helper type 2 profile (T_{H}2), existing in lesional and non-lesional skin, indicating a systemic switch to a T_{H}2-weighted profile (Sidbury, R., et al. Current allergy and asthma reports. 2017;17(7):42). An ideal atopic dermatitis treatment would thus address all of these underlying causes simultaneously, i.e. skin barrier deficiency, dysbiosis, and an activated cutaneous immune response. The present invention addresses these causes.

Engineered probiotics are a novel approach based on leveraging the skin microbiome for therapeutic purposes. Notably, an engineered probiotic has important advantages over other methods of drug delivery, as it will establish residence on the patient's skin and continuously and stably deliver therapeutic proteins in situ. Furthermore, certain strains of Staphylococcus epidermidis (SE) have demonstrated important beneficial immuno-modulatory and anti-pathogen effects in the skin, which are relevant to atopic dermatitis disease phenotype and severity. Moreover, the delivery of filaggrin, which is a structural protein derived from profilaggrin, further enhances the therapeutic approach due to filaggrin's role in the skin barrier and ability to reduce transepidermal water loss and improve skin hydration. The present invention has the surprising advantage of providing methods and compositions for treating skin diseases, e.g., atopic dermatitis, using a genetically engineered, recombinant strain of Staphylococcus epidermidis as a skin drug delivery system that secretes human filaggrin to address the pathophysiology of atopic dermatitis (e.g., AZT-01). Once applied to the skin, stable colonization of the skin and the subsequent secretion of filaggrin in situ can resolve the disease. The benefits of this invention include its safety as a non-steroidal treatment option, its efficacy due to the invention's combination of benefits from the secretion of filaggrin along with the benefits of the topical application of Staphylococcus epidermidis, and its ability to be therapeutically effective at even a low frequency of application (no more than once a day).

The present invention therefore addresses the long-felt need for an effective treatment for inflammatory skin diseases, such as atopic dermatitis. The present invention is also one of the first reported demonstrations of commensal skin bacteria that can secrete therapeutic proteins to treat skin disease.

### SUMMARY OF THE INVENTION

The invention refers to methods and compositions for treating inflammatory skin diseases comprising, as an active principle, an engineered microorganism capable of expressing therapeutically relevant recombinant fusion polypeptides (i.e. proteins, peptides, or amino acids).

The present invention features, in a first aspect, a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide. In a related embodiment, the recombinant microorganism further comprising a third coding sequence comprising a gene capable of expressing an export signal. In yet another embodiment, the expression of the first coding sequence, second coding sequence and third coding sequence is under the control of a promoter. In other embodiments, the arrangement of the first coding sequence, second coding sequence and third coding sequences are in-frame. In yet another related embodiment, the first coding sequence, second coding sequence and third coding sequence are operably linked to a promoter. In one embodiment, the recombinant microorganism is bacteria, or a combination of bacteria. In another embodiment, the polypeptide is filaggrin, or a variant thereof. In other embodiments, the microorganism is selected from the group consisting of Bifidobacterium, Brevibacterium, Propionibacterium, Lactococcus, Streptococcus, Staphylococcus, Lactobacillus, Enterococcus, Pediococcus, Leuconostoc, or Oenococcus, or combinations thereof. In yet other embodiments, the recombinant microorganism is Staphylococcus epidermidis. In another embodiment, the microorganism secretes a filaggrin fusion protein. In one embodiment, the polypeptide comprises an amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8 and SEQ ID NO: 9. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 1. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 2. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 3. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 4. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 5. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 6. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 7. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 8. In one embodiment, the polypeptide has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 9.

The present invention features, in a further aspect, a method for producing a live biotherapeutic composition, the method comprising (a) transfecting a cell with (i) a first coding sequence comprising a nucleic acid sequence capable of expressing a therapeutic polypeptide, and (ii) a second coding sequence comprising a nucleic acid sequence capable of expressing a cell penetrating peptide; and (b) allowing the transfected cell to produce a therapeutic polypeptide fusion protein; and (c) obtaining the live biotherapeutic composition. In a related embodiment, the method further comprises (iii) transfecting the cell with a third coding sequence comprising a nucleic acid sequence capable of expressing an export signal. In another embodiment, the first coding sequence, second coding sequence and third coding sequences are arranged in a single plasmid. In yet another embodiment, the arrangement of the first coding sequence, second coding sequence and third coding sequences are operably linked to a promoter. In other embodiments, the cell is selected from the group consisting of wherein the microorganism is selected from the group consisting of Bifidobacterium, Brevibacterium, Propionibacterium, Lactococcus, Streptococcus, Staphylococcus, Lactobacillus, Enterococcus, Pediococcus, Leuconostoc, or Oenococcus, or combinations thereof. In yet another embodiment, the cell is Staphylococcus epidermidis. In other embodiments, the therapeutic polypeptide fusion protein is a filaggrin fusion protein, or a variant thereof.

The present invention features, in another aspect, a nucleic acid comprising a nucleic acid sequence encoding a polypeptide as set forth any one of the aspects or embodiments herein.

The present invention features, in a further aspect, a composition obtained by any one of the method disclosed or described herein. In a related embodiment, the composition comprises a pharmaceutically acceptable carrier, wherein the pharmaceutically acceptable carrier is selected from the group consisting of an aqueous solution, an emulsion, a cream, a lotion, a gel, or an ointment.

The present invention features, in a further aspect, a live biotherapeutic composition comprising a recombinant microorganism wherein the recombinant microorganism comprises (i) a first coding sequence comprising a nucleic acid sequence capable of expressing a therapeutic polypeptide; (ii) a second coding sequence comprising a nucleic acid sequence capable of expressing a cell penetrating peptide; (iii) a third coding sequence comprising a nucleic acid sequence capable of expressing an export signal; and (iiv) a promoter operably linked to the first coding sequence, the second coding sequence and the third coding sequence; wherein the first coding sequence, second coding sequence and first coding sequence is capable of expressing a filaggrin fusion product, or variant thereof. In a related embodiment, the recombinant microorganism is Staphylococcus epidermidis. In a further embodiment, the export signal exports the filaggrin fusion product, or variant thereof, out of the recombinant microorganism. In yet another embodiment, the cell penetrating peptide facilitates the entry of the filaggrin fusion product, or variant thereof, into a human keratinocyte. In another embodiment, the composition comprises a pharmaceutically acceptable carrier, wherein the pharmaceutically acceptable carrier is selected from the group consisting of an aqueous solution, an emulsion, a cream, a lotion, a gel, or an ointment.

The present invention features, in a further aspect, a kit comprising any one of the compositions disclosed or described herein and instructions for use.

The present invention features, in a further aspect, a method of treating a skin disease comprising administering to a subject in need thereof the composition of any one of the compositions disclosed or described herein. In another embodiment, the skin disease is atopic dermatitis.

### BRIEF DESCRIPTION OF THE DRAWINGS

FIG. 1A-F depicts the relationship between staphylococcal species and atopic dermatitis. FIG. 1A shows longitudinal trend of mean proportion of S. aureus in atopic dermatitis in antecubital and popliteal creases (AcPC, n=12) grouped by no-treatment (trt) and intermittent-trt flares. FIG. 1B shows proportion of S. aureus and Shannon diversity index in AcPc. Partial correlation (adjusting for disease state, AcPc). FIG. 1C shows longitudinal trend of mean proportion of S. epidermidis in AcPc. FIG. 1D shows correlation of proportion of S. aureus versus objective SCORAD for each site (AcPc, Volar forearm [Vf], Nares [N]). Partial correlation (adjusting for disease state). FIG. 1E shows longitudinal Shannon diversity trend in atopic dermatitis grouped by no-treatment and intermittent-trt flares (n=12, Ac). FIG. 1F atopic dermatitis microbiome progression hypothesis. (*) Proposed relationship among shifts in skin microbial diversity, the proportion of Staphylococcus, and disease severity.

FIG. 2A depicts a schematic drawing of a Staphylococcus epidermidis-based protein delivery system, as described herein. The construct design comprises a promoter, a ribosome binding site (RBS), an export signal, a filaggrin expression sequence, and a cell-penetrating peptide sequence. FIG. 2B displays the characterization of certain promoters for tunable control of protein expression (using GFP as a reporter). FIG. 2C shows characterization of export signals for protein export out of SE (GFP used as reporter). FIG. 2D shows Western blot analysis of human filaggrin produced from Staphylococcus epidermidis, mouse filaggrin produced from Staphylococcus epidermidis, and whole mouse skin (anti-mouse flaggrin antibodies were used).

FIG. 3A-3D depicts the characterization of GFP-producing Staphylococcus epidermidis in reconstituted human epidermis (RHE). FIG. 3A shows fluorescence and light wavelength overlays of RHE at ˜25 μm at two hours past application of Staphylococcus epidermidis-GFP. FIG. 3B-3D shows fluorescence and light wavelength overlays of RHE two hours after dermaroller application then topical Staphylococcus epidermidis-GFP application. Depths taken at 0 μm FIG. 3B, 50 μm FIG. 3C, and 70 μm FIG. 3D.

FIG. 4A-4E depicts the characterization of SE-GFP colonization in mice. FIG. 4A-4C shows in vivo two-photon microscopy of mouse skin three days following treatment of GFP-expressing S. epidermidis. FIG. 4A 25 μm; and FIG. 4B 50 μm depth of unshaved mouse ear skin, and FIG. 4C 80 μm depth on shaved dorsal skin. FIG. 4D Light microscopy of dorsal skin of mice following SE-GFP application. FIG. 4E Light microscopy of dorsal skin of mice following SE-GFP application.

FIG. 5A-5N depicts the characterization of protein with and without the RMR signal using 50 μg GFP as a reporter in RHE. FIG. 5A-5D show two-photon images of topically applied GFP with (FIG. 5C, FIG. 5D) or without (FIG. 5A, FIG. 5B) the RMR signal at 30 minutes (FIG. 5A, FIG. 5C, FIG. 5E, FIG. 5G) or 60 minutes (FIG. 5B, FIG. 5D, FIG. 5F, FIG. 5H). Images are compiled Z-stacks projected onto a 2D plane. (FIG. 5E-FIG. 5H) 3D surface analysis to examine depth of protein penetration into the RHE. (FIG. 5I-FIG. 5N) Confocal images of GFP (FIG. 5K, FIG. N), GFP+RMR (FIG. 5J, FIG. 5M) or vehicle (FIG. 5I, FIG. 5L) using light (FIG. 5L-FIG. 5N) or fluorescent (FIG. 5I-FIG. 5K) wavelengths.

FIG. 6 depicts the experimental outline of 16S rRNA sequencing. FIG. 6 discloses SEQ ID NOS 30-33, 33-35, 35-36, and 36-42, respectively, in order of appearance.

FIG. 7 is a graph that shows hydrophobicity score as a function of amino acid position for the entire human filaggrin (hFLG) sequence (Uniprot P20930).

FIG. 8 is a graph that shows hydrophobicity score as a function of amino acid position for the entire mouse filaggrin (mFLG) sequence (NCBI Reference Sequence: XP_017175331.1).

FIG. 9 is a graph that shows hydrophobicity score as a function of amino acid position for the hFLG region starting at amino acid 1400 to 1800 with the unit segment of domains 9 and 10.

FIG. 10 is a graph that shows hydrophobicity score as a function of amino acid position for hFLG[9-10](1429-1774) from amino acid position 1400 to amino acid position 1800 in hFLG.

FIG. 11 is a graph that shows hydrophobicity score as a function of amino acid position for the start and end positions of the hFLG [9-10] (1429-1777).

FIG. 12 is a graph that shows hydrophobicity score as a function of amino acid position from a point of low hydrophobicity to the next point of low hydrophobicity (indicated by arrows).

FIG. 13 shows an alignment of human filaggrin dimers hFLG[3-4] (SEQ ID NO: 43), hFLG[5-6] (SEQ ID NO: 44), hFLG[7-8] (SEQ ID NO: 45), hFLG[9-10] (SEQ ID NO: 46), hFLG[11-12] (SEQ ID NO: 47), hFLG[13-14] (SEQ ID NO: 48), hFLG[15-16] (SEQ ID NO: 49), hFLG[17-18] (SEQ ID NO: 50), hFLG[19-20] (SEQ ID NO: 51), hFLG[21-22] (SEQ ID NO: 52).

FIG. 14 is a graph that shows background FLG binding at 1 μg/well for 2 h at 37° C. (non specific binding—NSB).

FIG. 15 is a graph that shows binding of hFLG segments to human callus keratin (NSB removed).

FIG. 16 is a graph that shows titration of IgY anti-hFLG.

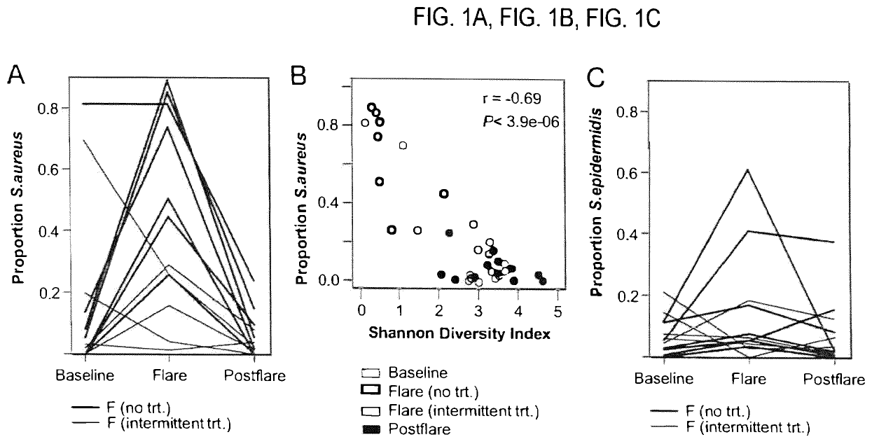

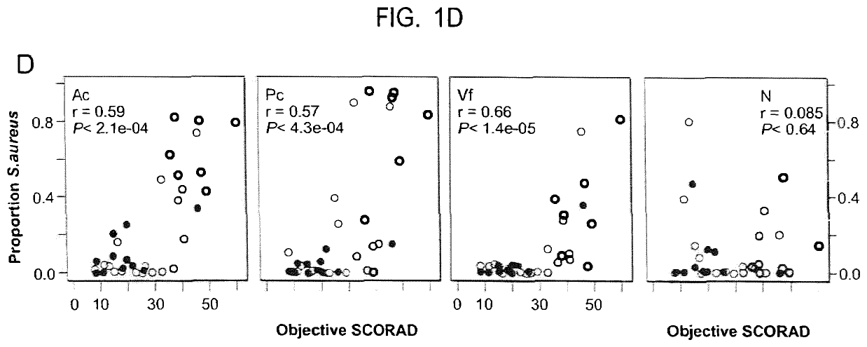

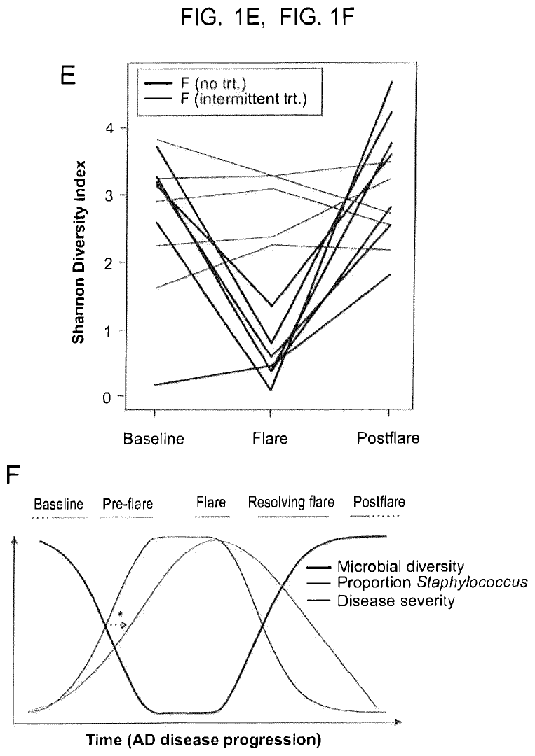

### DETAILED DESCRIPTION OF THE INVENTION

### I. Overview and Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The articles “a” and “an” are used herein to refer to one or to more than one (i.e. to at least one) of the grammatical object of the article. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

As used herein, the following terms have the following meanings unless expressly stated to the contrary: As used herein, the term “abnormal skin condition” or a “skin disease” (e.g., an inflammatory skin disease) refers to a skin state or condition that is generally undesirable or deleterious compared to the normal or baseline condition of human skin. Examples of abnormal skin conditions include: psoriasis, acne, atopic dermatitis, allergic contact dermatitis, epidermolytic hyperkeratosis, seborrheic dermatitis, eczema, dry skin, allergy, rashes, UV-irritated skin, detergent irritated skin (including irritation caused by enzymes and molecules used in washing detergents and sodium lauryl sulfate), thinning skin (e.g. skin from the elderly and children), bullous pemphigoid, pemphigus vulgaris, impetigo, vitiligio, baldness, and hirsutism.

As used herein, the terms “patient” or “subject”, refers to a human or animal (in the case of an animal, more typically a mammal such as domesticated mammals, or animals such as poultry animals and fish and other seafood or freshwater food creatures), that would be subjected to the treatments and compositions of the present invention. Such patient or subject would be considered to be in need of the pharmaceutical compositions of the present invention or of the methods of treating, preventing, or reducing the risk of an abnormal skin condition or a skin disease (e.g., an inflammatory skin disease).

As used herein, the term “therapeutically effective amount” refers to an amount of a pharmaceutical active compound, a live biotherapeutic composition, a combination of compounds or compositions, or an amount of pharmaceutical active compound delivered by an engineered bacterial strain or strains, for example a skin treatment agent or agents, when administered alone or in combination, to treat, prevent, or reduce the risk of a disease state or condition, for example an abnormal skin condition or a skin disease (e.g., an inflammatory skin disease). The term also refers to an amount of a pharmaceutical composition containing an active compound or combination of compounds or an engineered bacterial strain or strains that delivers a pharmaceutical active compound. For example, an effective amount refers to an amount of the compound or an amount of the compound delivered by an engineered bacterial strain (or a recombinant bacterial strain) or strains present in a formulation given to a recipient patient or subject sufficient to elicit biological activity, for example, activity for treating or preventing an abnormal skin condition or a skin disease (e.g., an inflammatory skin disease).

As used herein, the phrase “pharmaceutically acceptable” refers to those active compounds, materials, engineered bacterial strain or strains, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit/risk ratio. As used herein, the term “treating” refers to providing a therapeutic intervention to cure or ameliorate an abnormal skin condition.

As used herein, the term “preventing”, refers to completely or almost completely stopping an abnormal skin condition from occurring, for example when the patient or subject is predisposed to an abnormal skin condition or at risk of contracting an abnormal skin condition. Preventing can also include inhibiting, i.e. arresting the development, of an abnormal skin condition.

As used herein, the term “reducing the risk of,” refers to lowering the likelihood or probability of an abnormal skin condition from occurring, for example when the patient or subject is predisposed to an abnormal skin condition or at risk of contracting an abnormal skin condition.

As used herein, the term “engineered bacterial strain,” or a “recombinant bacterial strain” refers to a strain of bacteria that has been “genetically modified” or “engineered” by the introduction of DNA prepared outside the organism into the bacterial strain. For example, the introduction of a plasmid containing new genes or other nucleic acid sequence(s) into bacteria will allow the bacteria to express those genes or other nucleic acid sequence(s). Alternatively, the plasmid containing new genes or other nucleic acid sequence(s) can be introduced to the bacteria and then integrated into the bacteria's genome, where the bacteria will express those genes or other nucleic acid sequence(s).

As used herein, the terms “carriers”, “carrier system” or “vehicles” refer to compatible substances that are suitable for delivering, containing, or “carrying” a pharmaceutical active ingredient or other materials for administration in a topically applied composition to a patient or subject. Carriers useful herein should be pharmaceutically acceptable. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue. Further examples of “carriers” include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

As used herein, the terms “polypeptide” or “protein” refer to biological molecules, or macromolecules composed of amino-acid residues bonding together in a chain. The definition of polypeptides used herein is intended to encompass proteins (generally higher molecular weight) composed of one or more long chains of amino acid residues and small peptides (generally lower molecular weight) of a few amino acids. In other embodiments, a single amino acid, although not technically a polypeptide, is also considered within the scope of the invention.

As used here, the term “live biotherapeutic product” (or LBP) refers to a product candidate(s) containing bacteria, yeast, and/or other microorganisms.

The term “isolated” for the purposes of the present invention designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

An “isolated nucleic acid molecule” (such as, for example, an isolated promoter) is one which is separated from other nucleic acid molecules which are present in the natural source of the nucleic acid. For example, with regard to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule in the genomic DNA of the organism from which the nucleic acid molecule is derived.

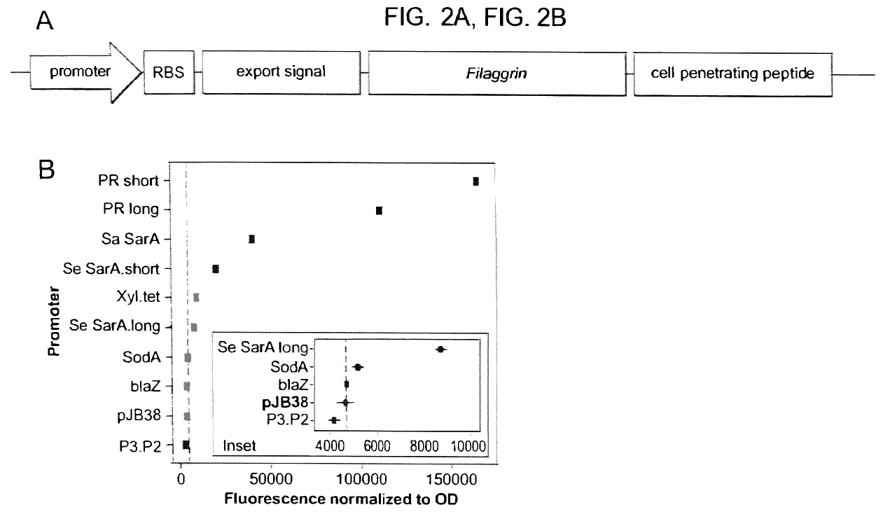

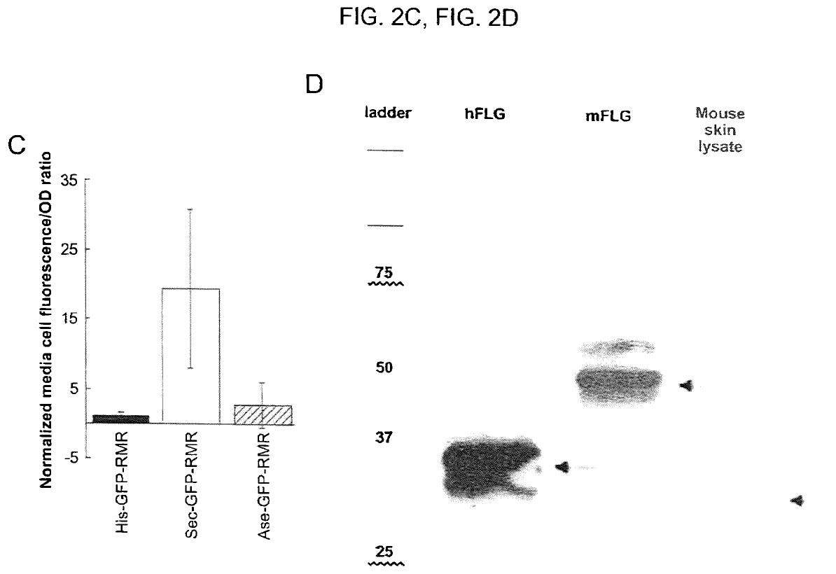

The present invention provides skin-colonizing bacteria that are genetically altered to express recombinant therapeutic polypeptides for the treatment or prevention of skin disease (FIG. 2). Using genetically engineered protein-producing bacteria has several advantages over the prior art method of treating skin disease. Therapeutic proteins are able to treat the underlying cause of defects leading to the skin condition. Further, bacteria are able to self-replicate while retaining the inserted gene to continuously produce the therapeutic protein.

The present invention provides skin-colonizing bacteria, such as for example, Staphylococcus epidermidis, that are genetically altered to express human filaggrin. Using genetically engineered filaggrin-producing bacteria has several advantages over using filaggrin supplementation. First, bacteria are able to self-replicate while retaining the inserted filaggrin gene. Second, S. epidermidis is normally present on the skin and has been shown to inhibit growth of Staphylococcus aureus, a bacterial species of the same genre that dominates the skin flora in AD flares.

### II. Methods and Compositions of the Invention

The present invention provides skin-colonizing microorganisms, e.g., bacteria, that are genetically altered to express recombinant therapeutic polypeptides for the treatment or prevention of skin disease (FIG. 2). Using genetically engineered protein-producing microorganisms, e.g., bacteria, has several advantages over the prior art method of treating skin disease. Therapeutic proteins are able to treat the underlying cause of defects leading to the skin condition. Further, microorganisms, e.g., bacteria, are able to self-replicate while retaining the inserted nucleic acid (e.g., a gene) to continuously produce the therapeutic protein.

The present invention provides skin-colonizing microorganisms, e.g., bacteria, such as for example, Staphylococcus epidermidis, that are genetically altered to express therapeutic proteins, e.g., human filaggrin. Using genetically engineered filaggrin-producing microorganisms, e.g., bacteria, has several advantages over using filaggrin supplementation. First, microorganisms, e.g., bacteria, are able to self-replicate while retaining the inserted filaggrin nucleic acid sequence (e.g., a gene). Second, S. epidermidis is normally present on the skin and has been shown to inhibit growth of Staphylococcus aureus, a bacterial species of the same genre that dominates the skin flora in atopic dermatitis flares.

### Bacterial Strains

The present invention provides genetically altered microorganisms, e.g., bacteria, capable of expressing recombinant therapeutic proteins. A wide range of microorganisms are suitable for use in the present invention. Examples include, but are not limited to, non-pathogenic and commensal bacteria. Bacteria suitable for use in the present invention include, but are not limited to, Bifidobacterium, Brevibacterium, Propionibacterium, Lactococcus, Streptococcus, Staphylococcus (e.g., S. epidermidis), Lactobacillus (e.g., L. acidophilus), Pediococcus, Leuconostoc, or Oenococcus. In certain embodiments of the invention, the bacterium is Staphylococcus epidermidis. In preferred embodiments of the invention, the strain of S. epidermidis to be used is incapable of producing biofilms. One such example of a strain of S. epidermidis incapable of producing biofilms is S. epidermidis strain ATCC 12228. However, in yet other embodiments of the invention, other related or similar species found on the skin can be used.

### Therapeutic Proteins

The present invention provides genetically altered microorganisms, e.g., bacteria, capable of expressing recombinant therapeutic proteins.

In some embodiments, the disclosure is directed to therapeutic proteins that include a filaggrin polypeptide amino acid sequence. In some embodiments, the disclosure is directed to therapeutic proteins that include a filaggrin polypeptide amino acid sequence and a cell penetrating polypeptide amino acid sequence. In some embodiments, the disclosure is directed to therapeutic proteins that include a filaggrin polypeptide amino acid sequence, a cell penetrating polypeptide amino acid sequence and a secretion signal or export signal polypeptide sequence. As used herein, a “polypeptide” generally is defined herein to refer to a peptide sequence of about 2 to about 10,000 or more amino acid residues. The term “amino acid” not only encompasses the 20 common amino acids in naturally synthesized proteins, but also includes any modified, unusual, or synthetic amino acid. One of ordinary skill in the art would be familiar with modified, unusual, or synthetic amino acids.

The polypeptides of the present invention may possess deletions and/or substitutions of amino acids relative to the native sequence; thus, sequences with a deletion, sequences with a substitution, and sequences with a deletion and a substitution are contemplated for inclusion in the polypeptides of the present invention. In some embodiments, these polypeptides may further include insertions or added amino acids, such as linkers.

Substitutional or replacement variants typically contain the exchange of one amino acid for another at one or more sites within the protein and may be designed to modulate one or more properties of the polypeptide, particularly to increase its efficacy or specificity. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

In addition to a deletion or substitution, the polypeptides may possess an insertion one or more residues. This may include the addition of one or more amino acid residues.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

### Filaggrin

In some embodiments, the disclosure is directed to therapeutic proteins that include a filaggrin polypeptide amino acid sequence. In preferred embodiments of the invention, the therapeutic protein comprises human filaggrin. Human filaggrin is expressed by a human gene encoding filaggrin (FLG). Filaggrin is a protein produced by differentiating keratinocytes and functions to aggregate keratin filaments into a cytoskeleton, which, in combination with other components, comprises the cornified cell envelope. FLG is a large gene located on chromosome lq21 that produces profilaggrin, an insoluble polyprotein that is proteolyzed to release functional filaggrin monomers (Armengot-Carbo et al. 2014). The therapeutic protein (and, i.e., the gene from which the protein is expressed) of the invention may be from any mammal. Non-limiting examples include, but are not limited to, mouse, rat, rabbit, goat, sheep, horse, cow, dog, primate, or human gene sequence.

The filaggrin amino acid sequences contemplated for inclusion in the polypeptides, compositions, and methods of the present invention may be obtained from any source. For example, the filaggrin amino acid may be obtained from a natural source or may be chemically synthesized. The filaggrin amino acid sequence may be from any species. For example, it may be a mammalian filaggrin amino acid sequence. Non-limiting examples include mouse, rat, rabbit, goat, sheep, horse, cow, dog, cat, primate, or human amino acid sequence. In preferred embodiments, the filaggrin amino acid sequence is a human amino acid sequence. Non-limiting examples of filaggrin proteins are set forth in Table 1, below.

TABLE 1

| Sequence | GenBank Accession No. | SEQ ID NO. |
|---|---|---|
| Filaggrin, Homo sapiens | NP_002007.1 | 1 |
| Filaggrin, Homo sapiens | AAA52454 | 10 |
| Filaggrin, Homo sapiens | P20930.3 | 11 |
| Filaggrin, Mus musculus | XP_017175331.1 | 12 |
| Filaggrin, Mus musculus | AAM23016 | 13 |
| Filaggrin, Mus musculus | AAA75559 | 14 |
| Filaggrin, Mus musculus | AAA37626 | 15 |
| Filaggrin, Mus musculus | XP_485270 | 16 |
| Filaggrin, Mus musculus | P11088 | 17 |
| Filaggrin, Mus musculus | EDL00668.1 | 18 |
| Filaggrin, Rattus norvegicus | EDL87862 | 19 |
| Filaggrin, Pan troglodytes | XP_001134714 | 20 |
| Filaggrin, Pan troglodytes | XP_513808 | 21 |
| Filaggrin, Bos taurus | XP_001255583 | 22 |
| Filaggrin, Macaca mulatta | XP_001101725.1 | 23 |
| Filaggrin, Macaca mulatta | XP_001109011.1 | 24 |

In some embodiments, the filaggrin amino acid sequence includes any of the amino acid sequences set forth in Table 1. In particular embodiments, the filaggrin amino acid sequence includes Gen Bank Accession No. NP_002007.1 (SEQ ID NO:1).

In some embodiments, the filaggrin amino acid sequence includes 20, 30, 40, 50, 60, 70, 80, 90, 100, 120, 130, 150, 150, 160, 170, 180, 190, 200, 210, 220, 230, 240, 250, 260, 270, 280, 290, 300, 310, 320, 330, 340 or more consecutive amino acids of any of the amino acid sequences set forth in Table 1, or any range of amino acids derivable therein, so long as the filaggrin amino acid sequence when conjugated to a cell penetrating peptide and/or export or secretion signal retains at least some of the function of a native filaggrin amino acid sequence conjugated to the same cell penetration peptide and/or export or secretion signal.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with a native filaggrin amino acid sequence, or any range of percent sequence identify derivable therein. In one embodiments, the filaggrin amino acid sequence is an amino acid sequence selected from Table 1. In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 1.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 2.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 3.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 4.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 5.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 6.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 7.

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 8.

In some embodiments, the human filaggrin consensus sequence is a consensus sequence shown as SEQ ID NO: 9, and refers to a sequence formed from the most frequently occurring amino acids in hFLG[3-4], hFLG[5-6], hFLG[7-8], hFLG[9-10], hFLG[11-12], hFLG[13-14], hFLG[15-16], hFLG[17-18], hFLG[19-20], hFLG[21-22].

SEQ ID NO: 9

| | |
|---|---|
| XLYQVSTHXQXDSXHGXTXXSTXXRQXSHXXQAXXXSRHSXSQXG | 100 |
| QDTIRGHPGXXXXGRQGXXXEXXVXXSGHSGXHHSHTTXQXRSDA | |
| SHGXSGXRSA | |
| SRXTXXXXQSXDXTRHSXSRHHEXXSXAXXSXHSXXGQXXSXGXR | 200 |
| XSRXXGSSXSQDXDSXXHSEDSERXSXSASRNHXGSXXEQXRXGS | |
| RXPXXHXEDR | |
| AXHGHSADXSRKSGTXHXXXSSXGQAASSXEQARSSXGERHGSRH | 300 |
| QXQSADSSXXSGXXHXQXSSAVXDSXXXGXSGSQATXXEGHSEDS | |
| DTQSVSGXGX | |
| XGXHQQSHXESXRXXSGXXSXRSXSFLY. | 328 |

In some embodiments, the filaggrin amino acid sequence has at least about 80%, 81%, 82%, 83, 84%, 85%, 86%, 87%, 88%, 89%, 90%, 91%, 92%, 93%, 94%, 95%, 96%, 97%, 98%, 99% or 100% sequence identity with SEQ ID NO: 9.

“Sequence identity” is defined as the percentage of amino acid residues in a candidate sequence that are identical with the amino acid residues at corresponding positions in a native polypeptide sequence, after aligning the sequences and introducing gaps if necessary, to achieve the maximum percent sequence identity, and not considering any conservative substitutions as part of the sequence identity. The % sequence identity values may be generated by the NCBI BLAST2.0 software as defined by Altschul et al. (1997). The parameters are set to default values, with the exception of Penalty for mismatch, which is set to −1.

In preferred embodiments of the invention, the therapeutic protein comprises a recombinant fusion protein comprising filaggrin operably linked to a cell penetrating protein (CPP). In other embodiments of the invention, the therapeutic protein comprises a recombinant fusion protein comprising filaggrin operably linked to an export or secretion signal, which allows the recombinant filaggrin to be exported out of the microorganism (e.g., bacteria). In another embodiment, the therapeutic protein comprises a recombinant fusion protein comprising filaggrin operably linked to a cell penetrating protein (CPP) and to an export or secretion signal.

Furthermore, the polypeptides set forth herein may comprises a sequence of any number of additional amino acid residues at either the N-terminus or C-terminus of the amino acid sequence that includes the filaggrin amino acid sequence and the cell penetrating protein (CPP) and/or export or secretion signal. For example, there may be an amino acid sequence of about 3 to about 10,000 or more amino acid residues at either the N-terminus, the C-terminus, or both the N-terminus and C-terminus of the amino acid sequence that includes the filaggrin amino acid sequence and the cell penetrating peptide and/or export or secretion signal.

### Secretion Signals

Secretion signals or export signals are peptide sequences on a protein that facilitate the export of the protein through the secretory pathway, which ultimately results in the protein being secreted from the cell. In the present invention, any secretion signal that facilitates the export of a protein, such as a protein comprising filaggrin, out of a microorganism (e.g., a bacterial cell) is contemplated as a secretion signal.

### Cell Penetrating Peptides

A cell penetrating peptide is a peptide sequence that facilitates or mediates the delivery of a biomolecule (e.g., a protein) in vivo without using any receptors and without causing any significant membrane damage. Cell penetrating peptides that facilitate entry into the skin keratinocytes are contemplated as a cell penetrating peptides of the present invention.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises a filaggrin polypeptide amino acid sequence.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 1.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 2.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 3.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 4.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 5.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 6.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 7.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 8.

According to some embodiments, the present disclosure provides a recombinant microorganism capable of secreting a polypeptide, wherein the recombinant microorganism comprises an expression vector comprising a first coding sequence comprising a gene capable of expressing the polypeptide and a second coding sequence comprising a gene capable of expressing a cell penetrating peptide, where the polypeptide comprises an amino acid sequence at least 95%, 96%, 97%, 98%, or 99% identical to SEQ ID NO: 9.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 2.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 3.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 4.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 5.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 6.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 7.

According to some embodiments, the present disclosure provides a recombinant microorganism capable of secreting a polypeptide, wherein the recombinant microorganism comprises an expression vector comprising an amino acid sequence at least 95%, 96%, 97%, 98%, or 99% identical to SEQ ID NO: 8.

### Nucleic Acids

The present invention includes nucleic acids that include a nucleic acid sequence that encodes a recombinant polypeptide of the present invention. Some embodiments of the present invention include a nucleic acid that includes a nucleic acid sequence that encodes a polypeptide as set forth above. Further embodiments include a nucleic acid that encodes a filaggrin amino acid sequence. The filaggrin amino acid sequence is any filaggrin amino acid sequence as set forth herein. In some embodiments, the nucleic acid is comprised in an expression vector. The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between 3 and about 100 nucleobases in length. The term “polynucleotide” refers to at least one molecule of greater than about 100 nucleobases in length.

These definitions refer to a single-stranded or double-stranded nucleic acid molecule. Double stranded nucleic acids are formed by fully complementary binding, although in some embodiments a double stranded nucleic acid may formed by partial or substantial complementary binding. Thus, a nucleic acid may encompass a double-stranded molecule that comprises one or more complementary strand(s) or “complement(s)” of a particular sequence, typically comprising a molecule. As used herein, a single stranded nucleic acid may be denoted by the prefix “ss” and a double stranded nucleic acid by the prefix “ds”.

### Genetic Construct

The present invention utilizes standard molecular biology techniques, e.g., those described in (Sambrook et al. 2001). An example of the genetic construct used for this invention is pAZT, which is based on pJB38, an allelic exchange E. coli-staphylococcal shuttle vector, further comprising additional design features on the plasmid to improve functionality (Bose, J. L., et al. Applied and environmental microbiology. 2013;79(7):2218-2224). The plasmid is constructed by inserting cDNA of a gene encoding a therapeutic protein into a restriction site, using standard molecular biology techniques (FIG. 2). The insert further comprises a coding sequence driven by a promoter. Such a promoter can be either constitutive or inducible. Examples of inducible promoters include those that are activated by chemical compounds such as alcohols, sugars, metals, or tetracycline, or by physical factors such as light or high temperatures.

The mRNA sequence of human FLG has a Genebank accession No. NM_002016. A plasmid pAZT was constructed by inserting part of the FLG cDNA into a restriction site of pJB38. The insert contains a nucleic acid coding sequence driven by a promoter. The construct further comprises a nucleic acid sequence encoding a secretion signal and a cell penetrating peptide, thus resulting in a recombinant filaggrin fusion protein.

### Uses of Recombinant Bacterial Strain

It will be understood that the skin disease to be treated can be any disease or disorder associated with skin. In preferred embodiments the disorder is selected from the group consisting of atopic dermatitis, psoriasis, acne, allergic contact dermatitis, epidermolytic hyperkeratosis, seborrheic dermatitis, eczema, dry skin, allergy, rashes, UV-irritated skin, detergent irritated skin (including irritation caused by enzymes and compounds used in washing detergents and sodium lauryl sulfate), thinning skin (e.g.

skin from the elderly and children), bullous pemphigoid, pemphigus vulgaris, impetigo, vitiligio, baldness, and hirsutism, Examples of proteins that can be administered according to the invention are preferably eukaryotic proteins. These proteins include, but are not limited to, single amino acids, small peptides, and large proteins. More particularly, genes encoding proteins that are useful in the invention as recombinant therapeutic proteins include, but are not limited to, the following: members of the interleukin family of genes, including, but not limited to, IL-2, IL-3, IL-4, IL-5, IL-6, IL-7, IL-8, IL-9, IL-10, IL-11, IL-12, IL-13, IL-14 and IL-15 and genes encoding receptor antagonists thereof. Genes which encode hematopoietic growth factors, including but not limited to, erythropoietin, granulocyte colony stimulating factor, granulocyte macrophage colony stimulating factor, macrophage colony stimulating factor, stem cell factor, leukemia inhibitory factor and thrombopoietin are also contemplated in the invention. Genes encoding neurotropic factors are also contemplated, including but not limited to, nerve growth factor, brain derived neurotropic factor and ciliary neurotropic factor. In addition, genes which encode interferons, including, but not limited, to IFN-alpha, IFN-beta and IFN-gamma are included. Further contemplated in the present invention are genes encoding chemokines such as the C—C family and the C—X—C family of cytokines, genes encoding hormones, such as proinsulin and growth hormone, and genes encoding thrombolytic enzymes, including tissue plasminogen activator, streptokinase, urokinase or other enzymes such as trypsin inhibitor. The invention further includes genes which encode tissue repair factors, growth and regulatory factors including, but not limited to, oncostatine M, platelet-derived growth factors, fibroblast growth factors, epidermal growth factor, hepatocyte growth factor, bone morphogenic proteins, insulin-like growth factors, calcitonin and transforming growth factor alpha and beta. Further contemplated genes include genes encoding structural proteins including filaggrin, actin, collagen, fibrillin, elastin, or scleroprotein.

### Formulations

It will be further apparent that a formulation for use according to the present invention may comprise any pharmaceutically effective amount of a genetically engineered microorganism, e.g., bacteria, to produce a therapeutically effective amount of a desired polypeptide, for example, at least about 0.01%, about 0.05%, about 0.1%, about 0.2%, about 0.3%, about 0.4%, about 0.5%, about 0.6%, about 0.7%, about 0.8%, about 0.9%, about 1.0%, about. 1.5%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0%, about 16.0%, about 17.0%, about 18.0%, about 19.0%, about 20.0%, about 25.0%, about 30.0%, about 35.0%, about 40.0%, about 45.0%, about 50.0% or more by weight of the genetically engineered microorganism, e.g., bacteria,, the upper limit of which is about 90.0% by weight of the genetically engineered microorganism, e.g., bacteria.

In an alternative embodiment, the formulation for use according to the present invention can comprise, for example, at least about 0,01% to about 30%, about 0.01% to about 20%, about 0.01% to about 5%, about 0.1% to about 30%, about 0.1% to about 20%, about 0.1% to about 15%, about 0.1% to about 10%, about 0.1% to about 5%, about 0.2% to about 5%, about 0,3% to about 5%, about 0.4% to about 5%, about 0.5% to about 5%, about 1% to about 5%, or more by weight of a genetically engineered microorganism, e.g., bacteria.

The topical formulation for use in the present invention can be in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter. The formulation can include a living biotherapeutic composition and can comprise at least one a genetically engineered microorganism, e.g., an engineered bacterial strain, that produces a recombinant polypeptide. This engineered living biotherapeutic composition can deliver the polypeptide directly to the skin for treating or preventing abnormal skin conditions, and/or skin diseases (e.g., inflammatory skin diseases).

Topical formulations include those in which any other active ingredients are dissolved or dispersed in a dermatological vehicle known in the art, e.g. aqueous or nonaqueous gels, ointments, water-in-oil or oil-in-water emulsions. Constituents of such vehicles may comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g. a mineral oil such as a liquid paraffin, natural or synthetic triglycerides such as MIGLYOL, or silicone oils such as dimethicone). Depending upon the nature of the formulation as well as its intended use and site of application, the dermatological vehicle employed can contain one or more components (e.g., when the formulation is an aqueous gel, components in addition to water) selected from the following: a solubilizing agent or solvent (e.g. a β-cyclodextrin, such as bydroxypropyl β-cyclodextrin, or an alcohol or polyol such as ethanol, propylene glycol or glycerol); a thickening agent (e.g. hydroxyethylceliulose, hydroxypropylcellulose, carboxymethylcellulose or carbomer); a gelling agent (e.g. a polyoxyethylene-polyoxypropylene copolymer); a preservative (e.g. benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt).

A pharmaceutically acceptable carrier can also be incorporated in the formulation of the present invention and can be any carrier conventionally used in the art. Examples thereof include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers. The term “pharmaceutically acceptable” or “pharmaceutically acceptable carrier” is used herein to refer to a compound or composition that can be incorporated into a pharmaceutical formulation without causing undesirable biological effects or unwanted, interaction with other components of the formulation, “carriers” or “vehicles” as used herein refer to carrier materials suitable for incorporation in a topically applied composition. Carriers and vehicles useful herein include any such materials known in the art, which are non-toxic and do not interact with other components of the formulation in which it is contained in a deleterious manner. The term “aqueous” refers to a formulation that contains water or that becomes water-containing following application to the skin or mucosal tissue.

Cream bases are water-washable, and contain an oil phase, an emulsifier, and an aqueous phase. The oil phase, also called the “internal” phase, is generally comprised of petrolatum and a fatty alcohol such ascetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions are preparations to be applied to the skin surface without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions are preferred formulations herein for treating large body areas, because of the case of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like. Solutions are homogeneous mixtures prepared by dissolving one or more chemical substances (solutes) in a liquid such that the molecules of the dissolved substance are dispersed among those of the solvent. The solution can contain other pharmaceutically or cosmetically acceptable chemicals to buffer, stabilize or preserve the solute. Common examples of solvents used in preparing solutions are ethanol, water, propylene glycol or any other acceptable vehicles. As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Preferred organic macromolecules, i.e., gelling agents, are cross-linked acrylic acid polymers such as the “carbomer” family of polymers, e.g., carboxypolyalkylenes that can be obtained commercially under the Carbopol trademark. Also preferred are hydrophilic polymers such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthaiate, and methylcellulose; gums such as tragacanth and xanthan gum; sodium alginate; and gelatin, In order to prepare a uniform gel, dispersing agents such as alcohol or glycerin can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, PA: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin, and hydrophilic petrolatum.

Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter δ of plasticizing enhancers is in the range of about 2.5 to about 10, preferably in the range of about 5 to about 10. Preferred lipophilic enhancers are fatty esters, fatty alcohols, and fatty ethers. Examples of specific and most preferred fatty acid esters include methyl laurate, ethyl oleate, propylene glycol nionolaurace, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C₂-C₄ alkane diol or triol, are substituted with one or two fatty ether substituents. Additional permeation enhancers will be known to those of ordinary skill in the art of topical drag delivery, and/or are described in the pertinent texts and literature. See, e.g., Percutaneous Penetration Enhancers, eds. Smith et al. (CRC Press, 1995) (incorporated herein by reference herein in its entirety).

Various other additives can be included in the compositions of the present invention in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in the formulations of the present invention are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylaury 1 ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), a-tocopherol (Vitamin E), β-tocopherol, γ-tocopherol, δ-tocopherol, ε-tocopherol, ζ_{ι}-tocopherol, Z^{Λ}-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the Carbopol trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhocic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants such as ascorbic acid (Vitamin C), a-tocopherol (Vitamin E), β-tocopherol, γ-tocopherol, 6-tocopherol, ε-tocopherol, ζ_{ι}-tocopherol, ζ₂-tocopherol, η-tocopherol, and retinol (Vitamin A), and/or pharmaceutically acceptable salts, esters, amides, or other derivatives thereof. A preferred tocopherol compound is α-tocopherol. Additional agents include those that are capable of improving oxygen supply in skin tissue, as described, for example, in Gross, et al., WO 94/00098 and Gross, et al., WO 94/00109, both assigned to Lancaster Group AG (incorporated herein by reference in their entirety). Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxyemnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene. See Title 21. Chapter 1. Subchapter D. Part 352. “Sunscreen drug products for over-the-counter human use” incorporated herein in its entirety. Other embodiments can include a variety of non-carcinogenic, non-irritating healing materials that facilitate treatment with the formulations of the invention. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The present invention contemplates amounts of these various additives equivalent to those conventionally used in the cosmetics field, and range, for example, from about 0.01% to about 20% of the total weight of the topical formulation.

The formulations of the invention can also include conventional additives such as opacifiers, fragrance, colorant, stabilizers, surfactants, and the like. In certain embodiments, other agents can also be added, such as antimicrobial agents, to prevent spoilage upon storage, i.e., to inhibit growth of microbes such as yeasts and molds.

Suitable antimicrobial agents for the present invention include, but are not limited to the following selected from the group consisting of the methyl and propyl esters of p-hydroxybenzoic acid (i.e., methyl and propyl paraben), sodium benzoate, sorbic acid, imidurea, and combinations thereof. In other embodiments, other agents can also be added, such as repressors and inducers, i.e., to inhibit (i.e., glycose) or induce (i.e. xylose) the production of the polypeptide of interest. Such additives can be employed provided they are compatible with and do not interfere with the function of the formulations.

The formulations can also contain irritation-mitigating additives to minimize or eliminate the possibility of skin irritation or skin damage resulting from the chemical entity to be administered, or other components of the composition.

Suitable irritation-mitigating additives include, for example: α-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than about 20 wt. %, more typically not more than about 5 wt. %, of the formulation. Further suitable pharmacologically active agents that can be incorporated into the present formulations in certain embodiments and thus topically applied along with the active agent include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites. Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil. A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally not involve more than one application per day. One of ordinary skill can readily determine the optimum amount of the formulation to be administered, administration methodologies and repetition rates. In general, it is contemplated that the formulations of the invention will be applied in the range of once or twice weekly up to once daily.

### III. Methods and Kits of the Invention

### Methods of Treatment

The invention provides methods for treating a skin disease, wherein the methods comprise administering to a subject in need of such treatment a genetically engineered microorganism, e.g., genetically engineered bacteria, capable of expressing a recombinant therapeutic fusion protein of the invention, thereby treating the subject. In a preferred embodiment, the disease is atopic dermatitis. In yet another preferred embodiment, the recombinant therapeutic fusion protein comprises filaggrin. In other embodiments, the recombinant therapeutic fusion protein comprises filaggrin operably linked to a cell penetrating peptide. In further embodiments, the recombinant therapeutic fusion protein is operably linked to an export signal.

### Kits

The present invention also provides kits. In one aspect, a kit of the invention comprises (a) a composition of the invention and (b) instructions for use thereof. In another aspect, a kit of the invention comprises (a) any one of the live biotherapeutic compositions of the invention, and (b) instructions for use thereof. Instructions can include an explanation of how to apply, administer, use, and maintain the compositions. The compositions of the invention are described supra. In some embodiments, a composition of the invention is an engineered microorganism capable of expressing therapeutically relevant recombinant fusion polypeptides, as described supra. In preferred embodiments, the composition comprises engineered bacteria (e.g., S. epidermidis) capable of expressing a recombinant fusion polypeptide comprising filaggrin.

In some embodiments, a kit can include a sealed container. Non-limiting examples of containers include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. Other examples of containers include glass or plastic vials or bottles. The kit and/or container can include indicia on its surface. The indicia, for example, can be a word, a phrase, an abbreviation, a picture, or a symbol.

The present invention is further illustrated by the following examples, which should not be construed as further limiting. The contents of all figures and all references, patents and published patent applications cited throughout this application, as well as the Figures, are expressly incorporated herein by reference in their entirety.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1: Development of a Nucleic Acid Construct That Can Encode a Protein Capable of Being Exported Out of the S. Epidermidis Cell and Then Imported Into Human Keratinocytes

The invention describes in one embodiment the generation of a recombinant S. epidermidis strains that is capable of heterologous protein secretion, therefore overcoming the intractability of genetic modification of S. epidermidis. Functional genetic analyses of the common skin colonizers S. aureus and S. epidermidis have previously been limited due to the presence of Type I and IV restriction systems in virtually all strains of these bacteria. These restriction systems recognize methylated cytosine bases in DNA from standard clone expansion systems such as DH10B E. coli. However, using a methylation deficient E. coli strain, DC10B, several constructs have been created in S. epidermidis strain ATCC12228⁵, which is a commensal, non-pathogenic isolate lacking ica operons implicated in S. epidermidis-associated catheter bloodstream infections. Accordingly, the invention describes the first known reported heterologous protein expression in S. epidermidis. The present invention thus provides, in one embodiment, a nucleic acid plasmid capable of encoding a protein that is exported out of the S. epidermidis cell and subsequently imported into human keratinocytes. This plasmid, pAZT, is based on pJB38 (Bose, J. L., et al. Applied and environmental microbiology. 2013;79(7):2218-2224), an allelic exchange E. coli-staphylococcal shuttle vector, which has been specifically re-engineered to possess features that improve functionality. The present invention provides, in one embodiment, an engineered S. epidermidis capable of effectively colonizing reconstituted epidermis and that is capable of producing 50 μg of protein per mL of ˜10⁹ CFU/mL (FIG. 2).

### Cell Penetrating Peptides (CPP)

Despite the formidable barrier properties of the epidermis, protein delivery through the stratum corneum has been demonstrated by employing transduction peptides or cell penetrating peptides (CPP). This challenge arises due to the diffusion impediments from the hydrophobic surface and the layers of linked corneocytes comprising the stratum corneum. However, by attaching a CPP sequence to the N-terminal end of a protein of interest, successful delivery of the fusion protein into the deeper epidermis is possible, in addition to the facilitation of intracellular localization and endosomal/lysosomal escape of the target protein. The present invention, in one embodiment, provides a construct that utilizes such an approach, and comprises an HIV trans-activator of a transcription-derived cell-penetration peptide (RMR) protein motif (FIG. 2). This approach is foundational to delivering protein into deeper layers of the skin for higher therapeutic effect.

### Safety and “Kill Switches”

A key requirement for nearly all recombinant microorganisms for clinical use is the ability to prevent undesired introduction to other individuals or environments. In order to ensure safety of the engineered strain, the present invention, in one embodiment, uses an auxotrophic strain, which requires supplementation of key amino acids (D-ala) or a certain metabolic gene (AlaR) for survival, and simultaneously replaces the need for an antibiotic resistant strain for selection, the latter of which is not commercially viable. In another embodiment, the present invention integrates a “kill switch”, which is based on CRISPR/Cas9 self-cleavage upon induction of a dual xylose-riboswitch promoter. In yet another embodiment, the present invention provides cell counters, which recombine out the AZT locus after a defined number of divisions, although this method would necessitate reapplication of the vehicle. To ensure the safety of the engineered S. epidermidis of the present invention, a CRISPR/Cas9-based kill switch, which is xylose-inducible and doubly regulated with a theophylline riboswitch, was developed. The basis of this approach is that Cas9 is extremely efficient at chromosomal cleavage given a targeting guide, and since staphylococci lack canonical non-homologous end joining repair pathways, genomic cleavage results in death in the absence of a homologous recombination template. The use of a CRISPR-based system also confers great specificity, since comparative genomics can be used to design guides unique to the engineered S. epidermidis strain of the present invention, such that the construct is inactive if spread to other microbes by horizontal gene transfer. Finally, in one embodiment, the present invention provides a construct designed to express multiple CRISPR spacers to simultaneously target multiple genomic regions to ensure cleavage and minimize survival by reversion.

### Example 2: Determine the Persistence and Localization of Topically Applied S. Epidermidis Using In Vitro Model Systems

### Materials and Methods

### Create Reporter Bacteria

In order to facilitate tracking of the topically applied bacteria, an sGFP-expressing strain of S. epidermidis (SE) was employed. The SecA and RMR peptides was removed such that the sGFP protein was not shuttled to the secretion system and free sGFP did not penetrate the stratum corneum. This construct is referred to as SE-sGFP.

### Quantify and Compare Growth Characteristics of Transformed Bacteria in Liquid Media

A basic understanding of the ability of transformed (recombinant) S. epidermidis to compete against wild type S. epidermidis was required. In order to understand the growth characteristics of the transformed bacteria and the growth dynamics of recombinant, protein-producing bacteria, standard techniques were used to quantify colony forming units (CFU) in liquid media. In order to determine growth differences between S. epidermidis-sGFP, S. epidermidis-chl and wild-type S. epidermidis, each strain was grown separately in two triplicate 100 mL cultures each for 12 hours. Every hour, a 1 mL sample was taken and measured at 395 nm and 600 nm to obtain measurements of both the signal of sGFP and the total concentration of bacteria, respectively. Fluorescence and optical density were compared across all samples to understand growth characteristics and sGFP production. The results indicated that protein production only slightly diminished the competitive growth of S. epidermidis-GFP relative to S. epidermidis-Chl as determined by fluorescence and CFU measurements.

### Quantification of the Growth of S. Epidermidis-GFP and Control Strains on RHE

In order to characterize the feasibility of applying bacteria to the skin, the growth dynamics of externally applied bacteria on an in vitro skin model was determined, with the understanding that this is only a first approximation of the ecological competition these bacteria would encounter on the skin of a human. Assays began two days after receiving the differentiated culture, in order to allow the culture to achieve stability after shipment. RHE cultures were established and maintained in antibiotic- and antifungal-free media (supplemented with Chl as needed) that were replaced every two days. Bacteria suspended in 50% glycerol were applied with a pipette to the center 3 mm diameter of the RHE. Control RHE with S. epidermidis-chl and S. epidermidis-WT bacteria were also applied and removed alongside the experimental arms. Upon removal from culture, the tissue inserts were homogenized and passed through a 5 μm filter to allow for collection of bacteria flow through. The bacterial suspensions were spun down, resuspended in media, and serially diluted and plated to determine the CFUs of bacteria in the insert. All measurements were normalized by the maximum recovery of bacteria as determined by the CFUs recovered 15 minutes after application.

### Qualitative Characterization of the Growth of SE-GFP and Control Strains on RHE

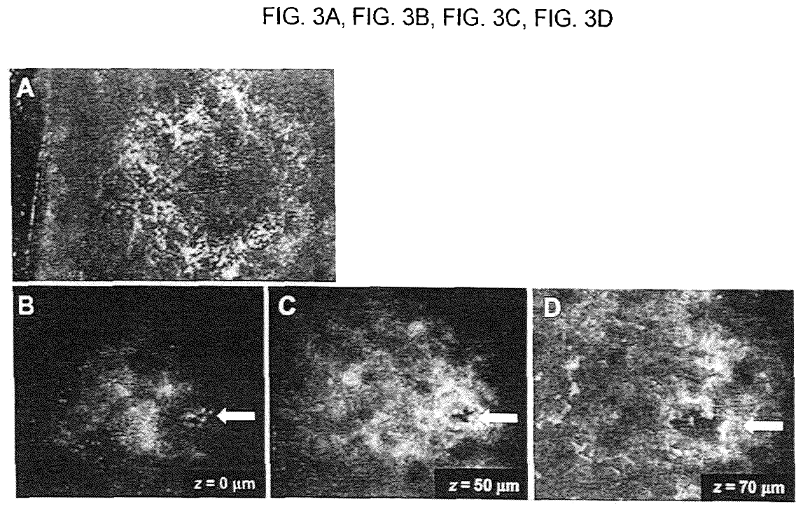

Assays were designed to obtain spatial and temporal information about S. epidermidis-GFP colonization using RHE and the Vivascope. S. epidermidis-GFP was applied to RHE, and samples were imaged in reflectance and fluorescence modes in three standardized regions 2 mm×2 mm wide and 100 μm deep using 10 μm steps and linear increase in laser power. Ultrasound gel (Parker Laboratories) was used to preserve the refractive index between the objective and the glass sample plate. Images were analyzed in ImageJ using ‘Grid/Collection Stitching’ (FIG. 3).

The results indicated that bacteria home to the surface and deep grooves of the stratum corneum layer and are maintained at a constant presence over the course of the experiment.

Importantly, in order to mimic the hyperstructure of damaged skin in atopic dermatitis patients, the RHE was intentionally punctured with a Derma Microneedle device to determine localization of the bacteria in the presence of damaged skin. The results indicated that the bacteria localized to the puncture at depths up to 70 μm (arrows, FIG. 3(B)-(D)). This suggests that the topically applied bacteria are able to hone to areas of damaged skin.

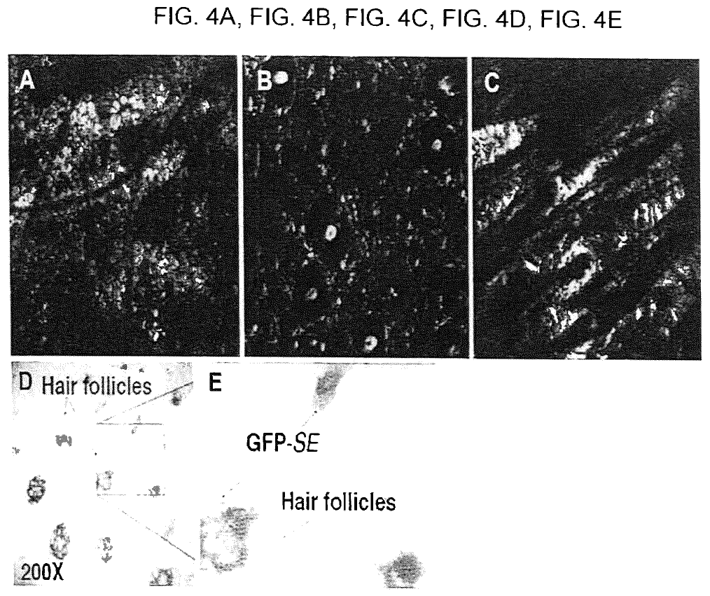

These studies were repeated in vivo. Specifically, SE-GFP was applied, to which light and in vivo two-photon microscopy was performed three days following application. At different depths, ranging from 25 μm in the mouse ear to 80 μm in shaved mouse dorsal skin (FIG. 4), the results indicated that there was sustained and pervasive GFP expression, demonstrating S. epidermidis-GFP's ability to colonize to the deepest layers of the stratum corneum (10-40 μm), and further colonize the hair follicles of the mice.

### Example 3: The Characterization of the Delivery of Bacterially Secreted sGFP to the Skin Using an In Vitro Model System

### Characterization of the Production of sGFP in SE

### Characterization of the Delivery of Bulk Purified sGFP and sGFP+RMR to RHE

Data regarding the localization of purified sGFP and sGFP+RMR would facilitate an understanding of: (i) whether sGFP+RMR penetrates the stratum corneum; (ii) if there is penetration, how deeply the penetration can be detected; and; (iii) the kinetic characteristics of penetration. Here, 5.0 μg/μL of GFP+/−RMR was applied at time points 0, 2, 6, 12, 18, and 24 hours to determine the effect of dosage on the penetration and time. The results indicated that GFP was detected as deep as the epidermal-dermal junction within 30 minutes after application.

### Characterization of the Cellular Compartment and Depth of Penetration of In Situ Secreted sGFP Protein From SE-GFP Reporter and Control Strains

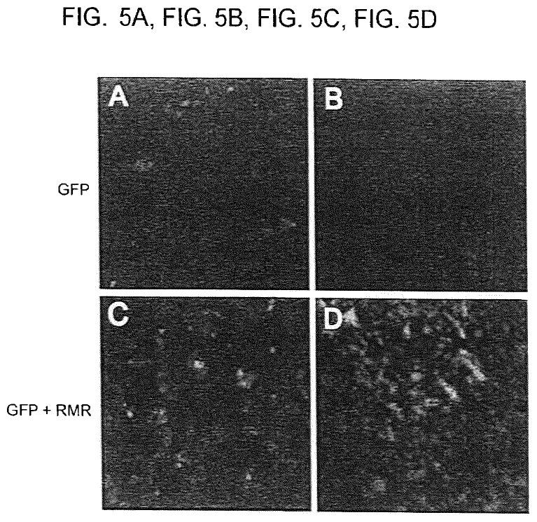

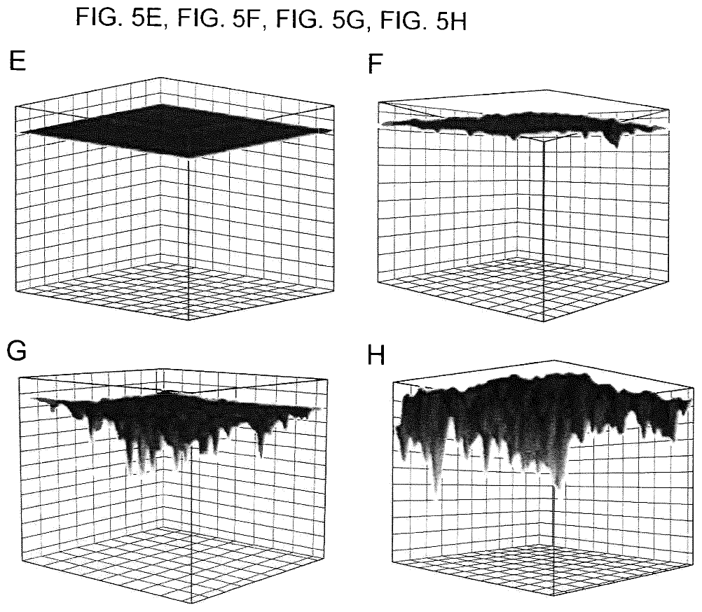

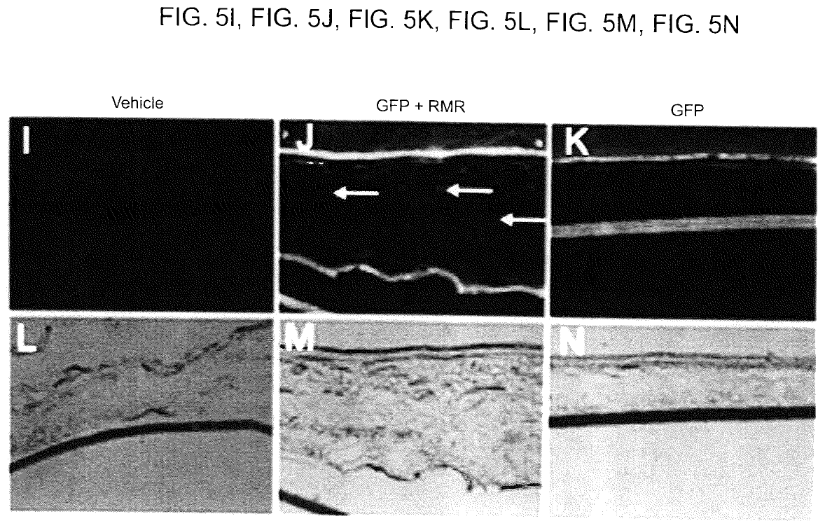

The goal of this assay is similar to that as described above, except for the critical difference that the protein is being made in situ by SE-sGFP^{RMR/SecA} and SE-sGFP^{SecA}, and compared to controls strains on the RHE. The same methods described herein were used to characterize the dynamics of sGFP penetration into the RHE. The Vivascope provided useful information on the penetration of sGFP over larger surface areas, while the use of IHC allowed for finer discrimination. IHC methods can detect GFP-positive regions relative to SE peptidoglycan-positive regions, so that secreted sGFP can be discriminated from the sGFP signal in the bacteria. (FIG. 5)

Non-fluorescence based detection of therapeutic proteins. The therapeutic proteins ultimately delivered are not be fluorescent, and further, suitable antibodies to characterize their delivery to epidermis may not be available. Proteomic analysis of tape strips of the stratum corneum has been used to characterize differences in in vivo protein profiles from patients with atopic dermatitis (Sakabe, J., et al. The Journal of allergy and clinical immunology. 2014;134(4):957-960 e958) and ichthyosis (Rice, R. H., et al. PloS one. 2013;8(10):e75355). Moreover, inside-out, horizontal sections of skin biopsies may be used to demonstrate that low abundance molecules can be identified after penetrating the stratum corneum.

### Example 4: Evaluate Pharmacokinetics (PK) and Pharmacodynamics (PD) of AZT-01 in Mice (Non-GLP)

A genetic atopic dermatitis mouse construct (flaky tail mice) was used to assess the PK/PD of AZT-01, while the PK of AZT-01 was assessed in healthy mice. In order to evaluate the PD, AZT-01 was topically applied to mice and the PD was assessed via phenotypic (erythema, edema, excoriation, dryness, and transepidermal water loss) and histological changes (skin barrier recapitulation, fibrosis, CD4+ T cells, etc.). To evaluate the PK, the distribution of AZT-01 and filaggrin was examined, and the colonization patterns were characterized. Changes in the skin microbiome were assessed.

Atopic dermatitis mouse models. In order to investigate the applicability of the approach, two model mouse systems were used: the filaggrin knockout mouse (flg−/−) and the flaky tail mouse (ft/ft). A thorough review of atopic the dermatitis model mice is presented by Geha et. al. (Vavrova K., et al. The Journal of Invest. Dermat. 2014;134(3):746-753.). Briefly, flg−/− mice exhibit dry, scaly skin. Despite marked decreases in natural moisturizing factor levels, which are filaggrin degradation products, stratum corneum (SC) hydration and transepidermal water loss (TEWL) were normal in flg−/− mice. Antigens penetrated the flg−/− SC more efficiently, leading to enhanced responses in hapten-induced contact hypersensitivity and higher serum levels of anti-ovalbumin IgG(1) and IgE. As such, the mouse ear was sensitized using OVA antigen.

Ft/ft mice possess two distinct autosomal recessive mutations, hair abnormality (matted: ma) and SC layer abnormality (flaky tail: ft). These mice developed dermatitis spontaneously with high serum IgE even under specific pathogen free conditions. Flaky tail mice also possess the loss of function mutation in Filaggrin (Flg) and demonstrated skin barrier abnormality as well as increased TEWL and SC hydration.

Study design. The study was conducted for four weeks using four arms in both types of mice (Flg−/− and ft/ft). Mice were randomized into the following treatment groups: topical vehicle control (50% glycerol, 50% sterilized BHI medium), topical recombinant filaggrin (purified recombinant filaggrin in 50 μg/ml), topical wild type Staphylococcus Epidermidis (SE) (1.0×10⁹ CFU in 50% glycerol), and topical SE^{FLG} (1.0×10⁹ CFU in 50% glycerol). Each solution was applied to the same ear on each mouse on days 0, 7, 14, and 21, and mice were also assessed on these days before application of the appropriate solution. Final assessment occurred on day 28 after which point the mice was sacrificed according to the appropriate animal protocols. The primary outcome (described in detail below) is the change in clinical disease score, which assessed macroscopic changes in disease presentation.

Based on a sample size estimate using standard deviation of 2.5, power of 90%, and type I error of 0.05, 8 mice per arm per genotype was required in order to detect a mean change of 4 points in the clinical disease score between groups. This means that a total of 32 mice per genotype (for a total of 64 mice) were needed for the study.

TABLE 2
Clinical outcome measures

| | | |
|---|---|---|
| Component | Values | Description |

Primary outcome: composite clinical disease score
(macroscopic observations

| | | |
|---|---|---|
| Erythema | 0 to 4 | 0-not visible, 2-mild, |
| Edema | 0 to 4 | 2-moderate, 3-severe |
| Excoriation | 0 to 4 | |
| Dryness | 0 to 4 | |
| Total: | 0 to 16 | Sum of four |
| | | components |

Secondary outcomes

| | | |
|---|---|---|
| Infiltrated | Qualitative | Cell types will be |
| lymphocytes | | indicated via histology |
| Fibrosis | 0 to 4 | 0-not visible, 2-mild, |
| | | 2-moderate, 3-severe |
| TEWL | Continuous | Measured by TEWL |
| | | meter |
| Skin barrier | Continuous | Measured by calcein |
| permeability | | assay |

Primary outcome: Clinical disease score. In order to measure the effect of treatment on the macroscopic and microscopic changes associated with treatment of SE^{FLG}, a clinical score based on previous studies was used (see, e.g., Matsuoka H., et al. Allergy. 2003;58(2):139-145, and Kim, M. C., et al. Journal of acupuncture and meridian studies. 2013;6(2):98-109). The composite clinical disease score is the sum of the degree of severity of erythema, edema, excoriation and dryness on the ear surface was scored as 0 (not visible), 1 (mild), 2 (moderate) and 3 (severe), accordingly. Scoring was performed by individual who was blinded to treatment status of each group. Skin was photographed every 7 days. Additionally, TEWL was measured by a TEWL meter (Khazaka Electronic). Finally, the methods described in Kawasaki (i.e., Kawasaki, H. et al. The Journal of allergy and clinical immunology. 2012;129(6):1538-1546.e1536) were used to assess the resolution of the skin barrier and its ability to prevent the permeation of foreign material past the stratrum corneum (SC). Calcein Bis[N,Nbis(carboxymethyl)aminomethyl] fluorescein (Sigma-Aldrich) was mixed with liposome prepared from Presome CSII-101 (Nippon Fine Chemical, Osaka, Japan) and topically applied to regions of the 6- to 8-week-old mice for 3 hours. The tails were then removed and rapidly freeze embedded.

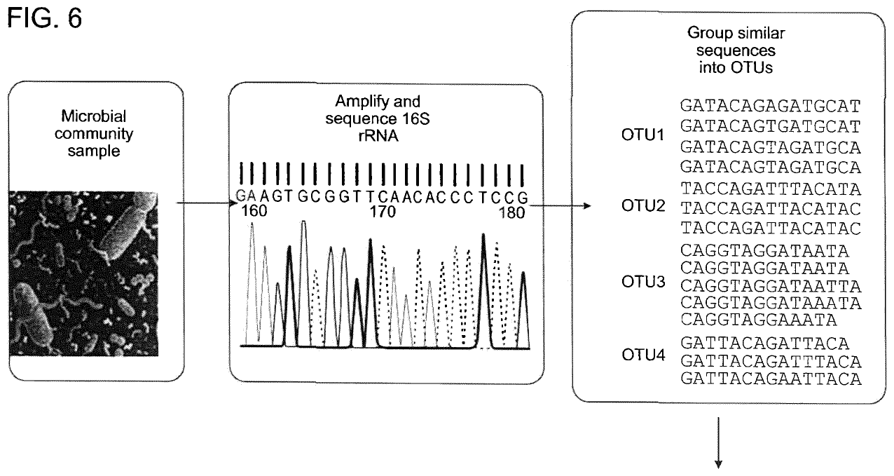

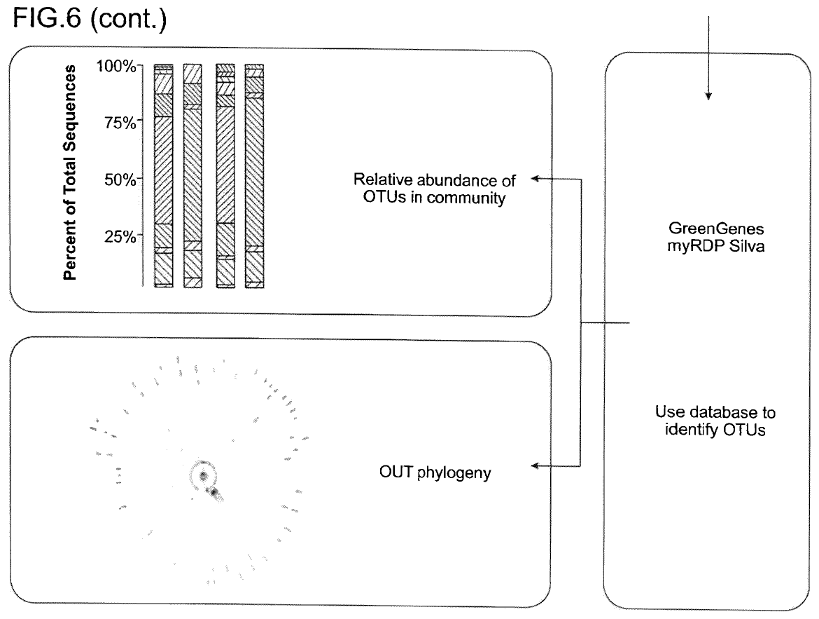

Microbiome characterization after application of AZT-01. In order to understand the influence of the addition of SE on the microbial diversity of the skin microbiome, a combined 16S rRNA was used to measure the changes in the microbial community. This was done using qPCR for RT-PCR and sequencing using the Illumina MiSeq platform at the JAX Genomic Medicine Facility at the Jackson Laboratory. The methods described in Caporaso et al. (Caporaso, J. G., et al. The ISME journal. 2012;6(8):1621-1624) were used to measure the changes in relative abundance of bacteria in the community. Briefly, samples were collected from the skin using cotton swabs and the rRNA was extracted using an rRNA extraction kit (Qiagen), which was then amplified, analyzed with qPCR and sequenced. Subsequently, bioinformatic and statistical methods were used to group similar sequences into operational taxonomic units (OTUs) (FIG. 6).

Dysbiosis was measured using ecological metrics and community structure analyses. First, dysbiosis was assessed as a function of diversity using the Shannon Diversity Index, which is an ecological measure of microbial communities that considers and was compared before and after application. Additionally, community structures of the local microbiome was compared before and after treatment. Dysbiosis was then measured as % overall deviation from (i) the baseline microbiome, and (ii) deviation from the mean community structure across our controls using statistics such as the Yue-Clayton index that compares community structures. Finally, microbiome trends was analyzed on a per-species level. The longitudinal dynamics of each species was also tracked over the treatments, to identify whether species are being lost from the community.

Immunohistochemistry studies. Filaggrin was visualized and quantified using immunocytochemistry by comparing AZT-01 to a vehicle control. Keratinocytes cells were fixed with 70% ethanol, 50 mM glycine for 1 hour. Immunofluorescence staining was performed by incubation of anti-filaggrin primary antibody at 1:200 for 2 hours, followed by incubation with rat anti-goat rhodamine secondary antibody (Jackson Laboratory) at 1:200 dilutions in the presence of Hoechst Stain Solution (Sigma). Slides were mounted with coverslips in Gel/Mount (Biomed). In addition, alternative sequences was created and tested in the place of the RMR signal (e.g., endosomal escape peptides such as those described in Appelbaum et al. 2012, incorporated by reference herein in its entirety (Appelbaum, J. S., et al. Chemistry & biology. 2012;19(7):819-830).

Statistical analyses. The differences between groups for the primary outcome and/or the macroscopic clinical disease score, were assessed using two-sided student T-tests. The differences across groups were assessed using ANOVA. The same technique will be used for assessing the TEWL and the thickening of the epidermis. The differences in non-parametric continuous variables were assessed using Mann-Whitney U tests. Finally, the differences in ordinal variables were assessed using Chi-square tests.

For analyses of the microbiome, community variation among samples were calculated using the quantitative, taxonomy-based Canberra distance. Discriminant analysis of within-group similarity were conducted using permutational MANOVA. To determine whether skin microbial communities became more similar to one another, we used a B-dispersion test with the betadisper function in vegan. This test is a multivariate analog of Levene's test for homogeneity of variances, and tests for a significant difference in sample heterogeneity between groups (Anderson, M. J., et al. Ecology Letters. 2006;9(6):683-693). P-values for significant indicators were adjusted for multiple comparisons using Holm's correction (Holm S. Scandinavian Journal of Statistics. 1979;6(2):65-70).

Immunofluorescence Microscopy. Immunofluorescence microscopy was used to visualize filaggrin localization. Mouse skin samples were fixed in 10% formalin and paraffin embedded. Paraffin sections were dewaxed and washed with 95% ethanol followed by methanol hydrogen peroxide. The sections were then treated with a heat induced epitope retrieval (HIER) procedure using rodent Decloaker solution (Biocare Medical, RD913) and the Biocare decloaking chamber. After being washed in Tris pH 7.4, sections were incubated in the presence of rat serum and FcBlock (24G2) followed by rabbit anti-Escherichia coli B (DAKO, B0357) diluted in the blocking solution. Samples were washed in Tris and then incubated with goat anti-rabbit IgG-Texas Red antibody (Invitrogen, T2767). The tissue was then counterstained with HOECSHT, and imaged using a Leica DM IRBE fluorescent microscope.

### Example 5: AZT-01 and its Effects on Local and Systemic Inflammation and Immunity in Rats (Non-GLP)

To evaluate toxicology, local tissue samples, serum samples, and lymph node samples of euthanized topically treated rats at specified time points were analyzed. The tissues were analyzed for histologic changes in inflammatory activity (e.g., quantification CD4+ T cells, Langerhans cells, IgE, IL-4, IFN-γ, etc.) as well as activity and changes in the cutaneous immune response (e.g., quantitation of IL-4, IL-10, IL-13, etc.). Clinical signs of potential side effects related to the use of therapy including erythema, skin temperature changes, edema, blistering, and ulcerations were also monitored.

Histological evaluation. Excised ears of each group were fixed in 4% paraformaldehyde for 16 h and were embedded in paraffin. Subsequently, 6 μm sections were stained with hematoxylin (Sigma Aldrich, St Louis, MO, USA) and eosin (Sigma Aldrich, St Louis, MO, USA) (H&E). Infiltrated lymphocytes and fibrosis in the dermis were observed by microscope (100×, 200×).

Disease-relevant mRNA transcript quantification. Variable expression of genes associated with atopic dermatitis development, progression, and maintenance (AD-associated pathogenic cytokines (e.g., IL-4, IL-5, IL-13, INF-γ, IL-17, IL-10 and TNF-α)) were measured by standard qPCR assays. Briefly, total RNA from the skin samples were isolated using the Qiagen RNeasy Mini Kit (Qiagen, Valencia, CA) following the manufacturer's instructions. The respective cDNA were synthesized using reverse transcriptase PCR (RT-PCR). Real-time PCR was performed using the comparative 2-ΔΔCT method and was normalized to Glyceraldehyde 3-phosphate dehydrogenase (GAPDH) transcript levels.

Immunological changes after application of AZT-01. Cells from areas of bacterial application as well as areas in which bacteria were not applied were isolated to study immunological changes. Keratinocytes, epidermal cells, cells from the lymph nodes, and small intestine lamina propria were isolated. Single cell suspensions were stained with either LIVE/DEAD Fixable Blue Dead Cell Stain Kit (Invitrogen) or with 4′,6-diamidino-2-phenylindol (DAPI, Sigma) in HBSS to exclude dead cells. For detection of transcription factors, cells were stained using the Foxp3 staining set (eBioscience) according to the manufacturer's protocol. For detection of intracellular cytokines, cells were fixed and permeabilized with BD Cytofix/Cytoperm and stained in BD Perm Wash buffer (BD Biosciences). Cells were stained with the following antibodies purchased from either eBioscience, BD Biosciences, or Dendritics Ccorp: CD4, IL-10, IL-17A, IFN-γ, TNF-α, Foxp3, CD34, CD44 and/or CD25. Staining was performed in the presence of FcBlock (eBioscience), 0.2 mg/ml purified rat IgG and 1 mg/ml of normal mouse serum (Jackson Immunoresearch). Stain for skin homing markers was performed as previously described (Lopes, L. B., et al. Pharm Res. 2005;22(5):750-757). As a measure of safety, signs of AZT-01 becoming blood-borne was monitored by taking samples of skin-draining lymph nodes (DLNs) and the spleen of one mouse after two weeks of application of AZT-01. Briefly, the mouse was placed in 70% EtOH and moved to a laminar flow hood. After 1-2 minutes in EtOH, DLNs and spleen was isolated in a 50-mL conical tube with a 70 μm strainer and was processed as separate samples. The organs were dissociated with 500 μl of sterile PBS, and 50-100 μl of cell suspension was cultured on BHI agar media.

### Example 6: Conduct Initial Formulation and Analytical Method Development to Evaluate a Proposed Set of Specifications for the Active Pharmaceutical Ingredient (API) and the Drug Product (DP)

Critical to the clinical development of a live biotherapeutic product (LBP; e.g., AZT-01) is developing a useful formulation and practical analytical assays given the unique nature of LBPs as active pharmaceutical ingredient (API) or drug product (DP) over traditional small molecules. A formulation will be developed for the API to produce the DP, and analytical methods will be developed for both the API and the DP stability to establish the specifications of GMP material for clinical studies.

Statistical analyses. Unless otherwise indicated, experiments were performed in triplicates, and means and standard deviations were be reported. For comparisons between groups, two-sided t-test or analysis of variance was used. If data were not normally distributed, the data were replaced with non-parametric equivalents (Wilcoxon-rank sum and Kruskal-Wallis tests).

### Example 7: Hydrophobicity Analysis

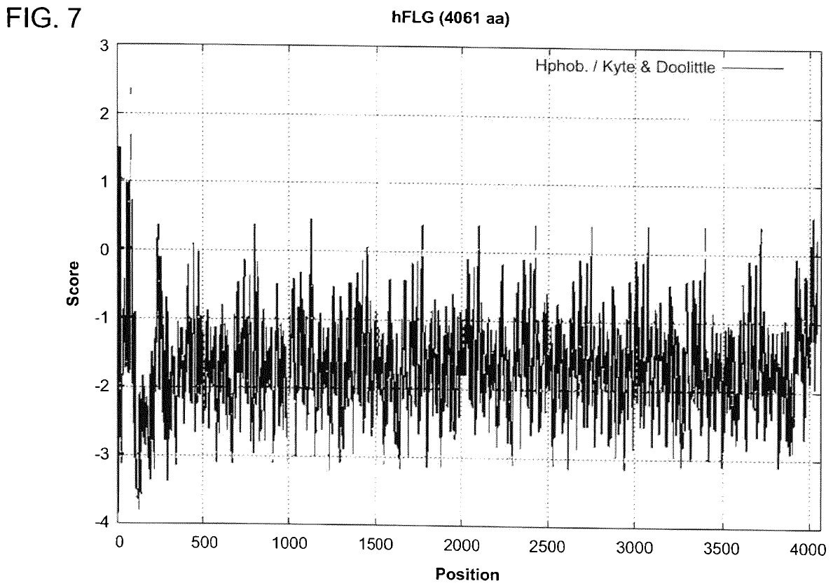

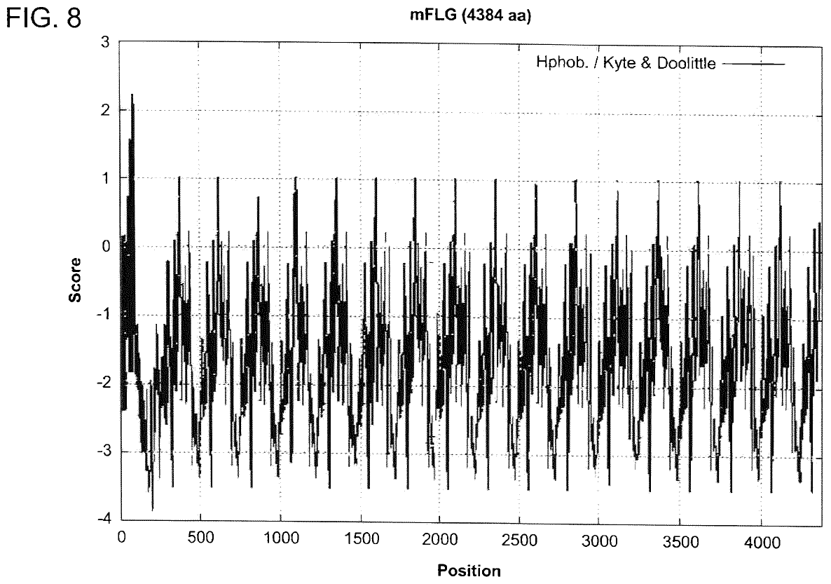

Human filaggrin (hFLG) is comprised of 12 repeating units and is processed by intracellular proteases to release the individual units. The sequences within these units are highly homologous. It has been proposed that the units are cleaved at “linker regions.” The segments between these linker regions are the individual units, with each unit containing a pair of sub-domains. The entire human filaggrin (hFLG) sequence (Uniprot P20930) was analyzed using the on-line Kite-Doolittle calculation tool (https://web.expasy.org/protscale/). The hydrophobicity score as a function of amino acid position graph is shown in FIG. 7. As shown in FIG. 7, there is a periodic increase in hydrophobicity. The Kite-Doolittle score gave the highest differential between the highest and lowest values. When compared to the mouse filaggrin (mFLG) sequence (NCBI Reference Sequence: XP_017175331.1) shown in FIG. 8, a much higher differential of hydrophobicity scores is seen. This is due to the nature of the proteins from the different organisms.

The molecular regions of interest were examined further. It was found that the distance between the crests which correspond to the more hydrophobic regions was farther in the human filaggrin than in the mouse. Also, the maximum hydrophobicity was found to be higher in the mouse than in human.

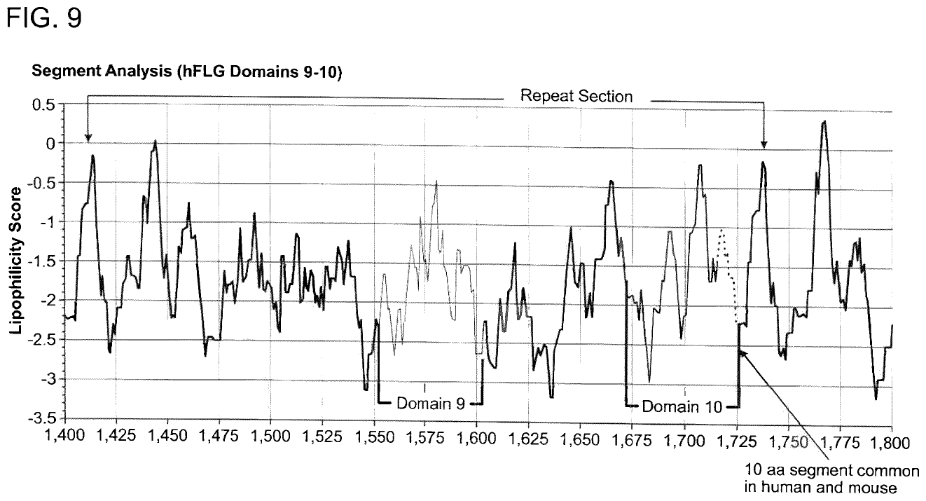

Two particular domains of hFLG were of interest, hFLG [domains 5-6] and [domains 9-10]. FIG. 9 shows the region of hFLG starting at amino acid 1400 to 1800, with the unit segment of domains 9 and 10 shown. The repeat end at a peak that comes right after the 10 amino acid segment that is common to human and mouse filaggrin (SGSQASDSEGHS) (SEQ ID NO: 25) is also shown. The high peaks at positions 1444 and 1767 of the graph correspond to the FLY segments. The peaks at 1679 and 1929 are regions containing poly-tyrosines.

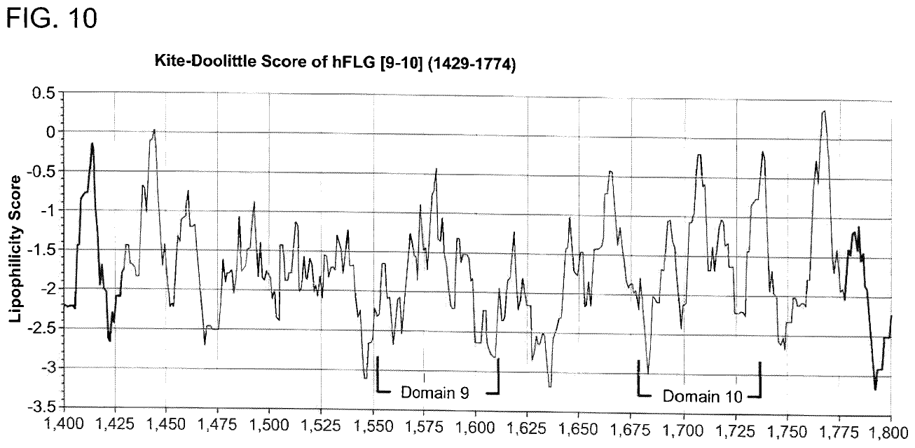

The protein hFLG (Domains 9-10) (1429-1774) (SEQ ID NO: 2) represents the high-to-high hydrophobicity segments. The segment is shown in FIG. 10. It contains the FLY segments at both ends of the protein.

```
hFLG[9-10](M-1429-1774)
SEQ ID NO: 2
                                                                                                    1430 1440
                                                                                                        MQ SGESSGRSRS
            1450 1460 1470 1480 1490 1500
FLYQVSSHEQ SESTHGQTAP STGGRQGSRH EQARNSSRHS ASQDGQDTIR GHPGSSRGGR
            1510 1520 1530 1540 1550 1560
QGSYHEQSVD RSGHSGYHHS HTTPQGRSDA SHGQSGPRSA SRQTRNEEQS GDGSRHSGSR
            1570 1580 1590 1600 1610 1620
HHEPSTRAGS SRHSQVGQGE SAGSKTSRRQ GSSVSQDRDS EGHSEDSERR SESASRNHYG
            1630 1640 1650 1660 1670 1680
SAREQSRHGS RNPRSHQEDR ASHGHSAESS RQSGTRHAET SSGGQAASSQ EQARSSPGER
            1690 1700 1710 1720 1730 1740
HGSRHQQSAD SSTDSGTGRR QDSSVVGDSG NRGSSGSQAS DSEGHSEESD TQSVSAHGQA
            1750 1760 1770 1780
GPHQQSHQES TRGQSGERSG RSGSFLYQVS THEQ
```

The complete sequence of the protein hFLG (Domains 9-10) (1429-1774) with N-terminus methionine and an RMR segment at C-terminus is shown in SEQ ID NO: 3 (M and RMR are underlined). The repeated sequence QSGEXSGRSXSFLYQVSXHEQSES (SEQ ID NO: 26) is shown in bold below.

```
hFLG[9-10](M-1429-1774-RMR)
SEQ ID NO: 3
                                                                                                    1430 1440
                                                                                                        MQ SGESSGRSRS
            1450 1460 1470 1480 1490 1500
FLYQVSSHEQ SESTHGQTAP STGGRQGSRH EQARNSSRHS ASQDGQDTIR GHPGSSRGGR
            1510 1520 1530 1540 1550 1560
QGSYHEQSVD RSGHSGYHHS HTTPQGRSDA SHGQSGPRSA SRQTRNEEQS GDGSRHSGSR
            1570 1580 1590 1600 1610 1620
HHEPSTRAGS SRHSQVGQGE SAGSKTSRRQ GSSVSQDRDS EGHSEDSERR SESASRNHYG
            1630 1640 1650 1660 1670 1680
SAREQSRHGS RNPRSHQEDR ASHGHSAESS RQSGTRHAET SSGGQAASSQ EQARSSPGER
            1690 1700 1710 1720 1730 1740
HGSRHQQSAD SSTDSGTGRR QDSSVVGDSG NRGSSGSQAS DSEGHSEESD TQSVSAHGQA
            1750 1760 1770 1780
GPHQQSHQES TRGQSGERSG RSGSFLYQVS THEQRMRRMR RMRR
```

It was found that repeating the FLY segments may not be required for activity, and further, it may be a burden for bacteria and may be the source of the lack of production in a Gram positive organism.

FIG. 10 is a graph that shows hydrophobicity score as a function of amino acid position for hFLG[9-10](1429-1774) from amino acid position 1400 to amino acid position 1800 in hFLG.

### Example 8. New Protein Structures Based on Hydrophobicity Analysis hFLG [Domains 9-10]

Next, hFLG [domain 9-10] proteins with systematically shaved off regions of the N-terminus were produced.

### Selection of Segment Based on “U” Shape (“High-Low-High”)

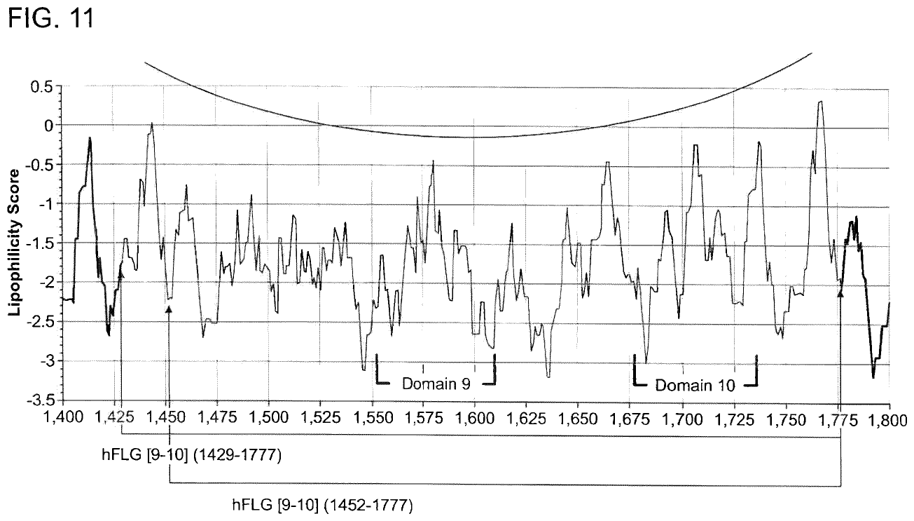

The graph in FIG. 11 shows that the start and end positions of the hFLG [9-10] 1429-1777) (SEQ ID NO: 2) may be too long. Therefore, the N-terminal FLY segment was cut to make hFLG [9-10] (1452-1777) (SEQ ID NO: 4) to prevent unwanted recombination events.

```
hFLG[9-10](1452-1777)-RMR
SEQ ID NO: 4
                                  1460 1470 1480 1490 1500
                      MESTHGQTAP STGGRQGSRH EQARNSSRHS ASQDGQDTIR GHPGSSRGGR
            1510 1520 1530 1540 1550 1560
QGSYHEQSVD RSGHSGYHHS HTTPQGRSDA SHGQSGPRSA SRQTRNEEQS GDGSRHSGSR
            1570 1580 1590 1600 1610 1620
HHEPSTRAGS SRHSQVGQGE SAGSKTSRRQ GSSVSQDRDS EGHSEDSERR SESASRNHYG
            1630 1640 1650 1660 1670 1680
SAREQSRHGS RNPRSHQEDR ASHGHSAESS RQSGTRHAET SSGGQAASSQ EQARSSPGER
            1690 1700 1710 1720 1730 1740
HGSRHQQSAD SSTDSGTGRR QDSSVVGDSG NRGSSGSQAS DSEGHSEESD TQSVSAHGQA
            1750 1760 1770
GPHQQSHQES TRGQSGERSG RSGSFLYQVS THEQSESRMR RMRRMRR
Theoretical pl/Mw: 10.65/36162.11
```

### Selection of Segment Based on “A” Shape (“Low-High-Low”)

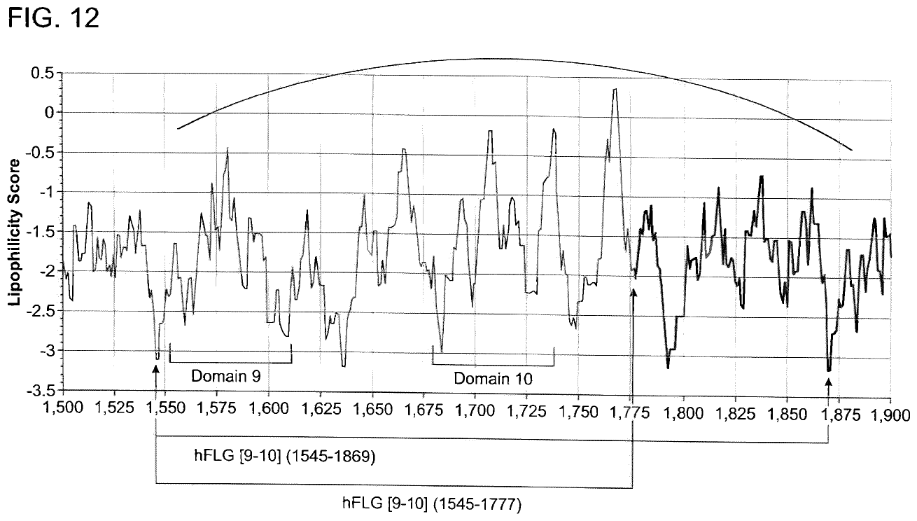

Next, the selection of a repeat sequence from a point of low hydrophobicity to the next point of low hydrophobicity was carried out. This segment has the two domains at the N-terminus with the FLY region close to the middle of the sequence. Thus, the “core” sequence containing the domains 9-10 are at an end, rather than in the middle of the sequence. Further, the long intersection repeat sequence is toward the C-terminus of the sequence. This is shown in FIG. 12.

Based on the aforementioned analyses, a protein based on the low-to-low segment hFLG[9-10](1545-1869) was made, shown below as SEQ ID NO: 5.

```
hFLG[9-10](1545-1869)-RMR
SEQ ID NO: 5
                                                                                                    1550 1560
                                                                                              MRNEEQS GDGSRHSGSR
            1570 1580 1590 1600 1610 1620
HHEPSTRAGS SRHSQVGQGE SAGSKTSRRQ GSSVSQDRDS EGHSEDSERR SESASRNHYG
            1630 1640 1650 1660 1670 1680
SAREQSRHGS RNPRSHQEDR ASHGHSAESS RQSGTRHAET SSGGQAASSQ EQARSSPGER
            1690 1700 1710 1720 1730 1740
HGSRHQQSAD SSTDSGTGRR QDSSVVGDSG NRGSSGSQAS DSEGHSEESD TQSVSAHGQA
            1750 1760 1770 1780 1790 1800
GPHQQSHQES TRGQSGERSG RSGSFLYQVS THEQSESAHG RTGPSTGGRQ RSRHEQARDS
            1810 1820 1830 1840 1850 1860
SRHSASQEGQ DTIRGHPGSS RGGRQGSHYE QSVDSSGHSG SHHSHTTSQE RSDVSRGQSG
            1870
SRSVSRQTRR MRRMRRMRR
Theoretical pl/Mw: 11.00/36182.19
```

The importance of the segment that has no known function will be evaluated by removing it in hFLG[9-10] (1545-1777), shown below as SEQ ID NO: 6.

```
hFLG[9-10](1545-1777)-RMR
SEQ ID NO: 6
                                                                                                    1550 1560
                                                                                              MRNEEQS GDGSRHSGSR
            1570 1580 1590 1600 1610 1620
HHEPSTRAGS SRHSQVGQGE SAGSKTSRRQ GSSVSQDRDS EGHSEDSERR SESASRNHYG
            1630 1640 1650 1660 1670 1680
SAREQSRHGS RNPRSHQEDR ASHGHSAESS RQSGTRHAET SSGGQAASSQ EQARSSPGER
            1690 1700 1710 1720 1730 1740
HGSRHQQSAD SSTDSGTGRR QDSSVVGDSG NRGSSGSQAS DSEGHSEESD TQSVSAHGQA
            1750 1760 1770
GPHQQSHQES TRGQSGERSG RSGSFLYQVS THEQSESRMR RMRRMRR
Theoretical pl/Mw: 10.07/26329.02
```

It is further contemplated that on hFLG[9-10](1452-1777) (SEQ ID NO: 2), the N-terminus region would be knocked off.

### Example 9. Filaggrin (M-AZT-Mutein-RMR)

The hFLG[9-10] sequence (SEQ ID NO: 1) was subjected to a BLAST analysis:

Alignment statistics for match #1

| Score | Expect | Method | Identities | Positives | Gaps |
|---|---|---|---|---|---|
| 625 bits(1611) | 0.0 | Compositional | 345/345(100%) | 345/345(100%) | 0/345(0%) |
| | | matrix adjust. | | | |

| | | | |
|---|---|---|---|
| Query | 1 | QSGESSGRSRSFLYQVSSHEQSESTHGQTAPSTGGRQGSRHEQARNSSRHSASQDGQDTI | 60 |
| | | QSGESSGRSRSFLYQVSSHEQSESTHGQTAPSTGGRQGSRHEQARNSSRHSASQDGQDTI | |
| Sbjct | 1430 | QSGESSGRSRSFLYQVSSHEQSESTHGQTAPSTGGRQGSRHEQARNSSRHSASQDGQDTI | 1489 |
| Query | 61 | RGHPGSSRGGRQGSYHEQSVDRSGHSGYHHSHTTPQGRSDASHGQSGPRSASRQTRNEEQ | 120 |
| | | RGHPGSSRGGRQGSYHEQSVDRSGHSGYHHSHTTPQGRSDASHGQSGPRSASRQTRNEEQ | |
| Sbjct | 1490 | RGHPGSSRGGRQGSYHEQSVDRSGHSGYHHSHTTPQGRSDASHGQSGPRSASRQTRNEEQ | 1549 |
| Query | 121 | SGDGSRHSGSRHHEPSTRAGSSRHSQVGQGESAGSKTSRRQGSSVSQDRDSEGHSEDSER | 180 |
| | | SGDGSRHSGSRHHEPSTRAGSSRHSQVGQGESAGSKTSRRQGSSVSQDRDSEGHSEDSER | |
| Sbjct | 1550 | SGDGSRHSGSRHHEPSTRAGSSRHSQVGQGESAGSKTSRRQGSSVSQDRDSEGHSEDSER | 1609 |
| Query | 181 | RSESASRNHYGSAREQSRHGSRNPRSHQEDRASHGHSAESSRQSGTRHAETSSGGQAASS | 240 |
| | | RSESASRNHYGSAREQSRHGSRNPRSHQEDRASHGHSAESSRQSGTRHAETSSGGQAASS | |
| Sbjct | 1610 | RSESASRNHYGSAREQSRHGSRNPRSHQEDRASHGHSAESSRQSGTRHAETSSGGQAASS | 1669 |
| Query | 241 | QEQARSSPGERHGSRHQQSADSSTDSGTGRRQDSSVVGDSGNRGSSGSQASDSEGHSEES | 300 |
| | | QEQARSSPGERHGSRHQQSADSSTDSGTGRRQDSSVVGDSGNRGSSGSQASDSEGHSEES | |
| Sbjct | 1670 | QEQARSSPGERHGSRHQQSADSSTDSGTGRRQDSSVVGDSGNRGSSGSQASDSEGHSEES | 1729 |
| Query | 301 | DTQSVSAHGQAGPHQQSHQESTRGQSGERSGRSGSFLYQVSTHEQ (SEQ ID NO: 27) | 345 |
| | | DTQSVSAHGQAGPHQQSHQESTRGQSGERSGRSGSFLYQVSTHEQ (SEQ ID NO: 28) | |
| Sbjct | 1730 | DTQSVSAHGQAGPHQQSHQESTRGQSGERSGRSGSFLYQVSTHEQ (SEQ ID NO: 27) | 177 |

This sequence was compared to the other hFLG units (FLG[3-4], hFLG[5-6], hFLG[7-8], hFLG[11-12], hFLG[13-14], hFLG[15-16], hFLG[17-18], hFLG[19-20], hFLG[21-22]). The amino acids that were variable were compared and the most common ones were replaced into the [9-10] sequence.

The new sequence (S-FLG) was compared to the original hFLG[9-10] sequence by multiple alignment, shown below:

| | | |
|---|---|---|
| hFLG[9-10] | MQSGESSGRSRSFLYQVSSHEQSESTHGQTAPSTGGRQGSRHEQARNSSRHSASQDGQDT | |
| S-FLG | M-------RSRSFLYQVSSHEQSESTHGQTAPSTGGRQGSRHEQARNSSRHSASQDGQDT | |
| | ************************************************************ | |
| hFLG[9-10] | IRGHPGSSRGGRQGSYHEQSVDRSGHSGYHHSHTTPQGRSDASHGQSGPRSASRQTRNEE | |
| S-FLG | IRGHPGSSRGGRQGSYHEQSVDRSGHSGYHHSHTTPQGRSDASHGQSGPRSASRQTRNEE | |
| | ************************************************************ | |
| hFLG[9-10] | QSGDGSRHSGSRHHEPSTRAGSSRHSQVGQGESSGSKTSRRQGSSVSQDRDSEGHSEDSE | |
| S-FLG | QSGDGSRHSGSRHHEASTRADSSRHSQVGQGQSSGSRTSRRQGSSVSQDSDSEGHSEDSE | |
| | ***************.****.**********:*:**:************ ********** | |
| hFLG[9-10] | RRSESASRNHYGSAREQSRHGSRNPRSHQEDRASHGHSAESSRQSGTRHAETSSGGQAAS | |
| S-FLG | RRSGSASRNHYGSAQEQSRDGSRHPRSHQEDRASHGHSAESSRQSGTRHAETSSGGQAAS | |
| | *** **********:****.***:************************************ | |
| hFLG[9-10] | SQEQARSSPGERHGSRHQQSADSSTDSGTGRRQDSSVVGDSGNRGSSGSQASDSEGHSEE | |
| S-FLG | SHEQARSSPGERHGSRHQQSADSSRHSGIGHGQASSAVRDSGHRGSSGSQASDSEGHSED | |
| | *:********************** .** *: * **.* ***:****************: | |
| hFLG[9-10] | SDTQSVSAHGQAGPHQQSHQESTRGQSGERSGRSGSFLYQVSTHEQ---RMRRMRRMRR | (SEQ ID NO: 3) |
| S-FLG | SDTQSVSAHGQAGPHQQSHQESARGRSGERSGRSGSFLYQVSTHEQSESRMRRMRRMRR | (SEQ ID NO: 8) |
| | **********************:**:******************** ********** | |

hFLG[M-AZT-mutein-RMR](1-352) was created, shown below as SEQ ID NO: 8.

```
hFLG[M-AZT-mutein-RMR](1-352)
              10 20 30 40 50 60
MRSRSFLYQV SSHEQSESTH GQTAPSTGGR QGSRHEQARN SSRHSASQDG QDTIRGHPGS
                70 80 90 100 110 120
SRGGRQGSYH EQSVDRSGHS GYHHSHTTPQ GRSDASHGQS GPRSASRQTR NEEQSGDGSR
              130 140 150 160 170 180
HSGSRHHEAS TRADSSRHSQ VGQGQSSGSR TSRRQGSSVS QDSDSEGHSE DSERRSGSAS
              190 200 210 220 230 240
RNHYGSAQEQ SRDGSRHPRS HQEDRASHGH SAESSRQSGT RHAETSSGGQ AASSHEQARS
              250 260 270 280 290 300
SPGERHGSRH QQSADSSRHS GIGHGQASSA VRDSGHRGSS GSQASDSEGH SEDSDTQSVS
              310 320 330 340 350
AHGQAGPHQQ SHQESARGRS GERSGRSGSF LYQVSTHEQS ESRMRRMRRM RR
```

The amino acids shown in bold in SEQ ID NO: 8 were modified to the most prevalent amino acid when comparing every FLG unit. Table 3 below shows the amino acid residue in hFLG[9-10] and the corresponding modified amino acid in hFLG[AZT-mutein]. The Ser-Glu-Ser (SES) was re-introduced in the sequence to align with the Kite-Doolittle analysis. The QSGESSG (SEQ ID NO: 9) sequence was removed for the same reason as the SES and was calculated to be frivolous.

TABLE 3

| | Amino Acid | |
|---|---|---|
| Position | in hFLG[9-10] | Amino Acid in hFLG[AZT-mutein] |
| 130 | P | S |
| 134 | G | D |
| 145 | E | Q |
| 147 | A | S |
| 150 | K | R |
| 163 | R | S |
| 177 | E | G |
| 188 | R | Q |
| 193 | H | D |
| 197 | N | H |
| 235 | Q | H |
| 258 | T | R |
| 259 | D | H |
| 262 | T | I |
| 264 | R | H |
| 265 | R | G |
| 267 | D | A |
| 270 | V | A |
| 272 | G | R |
| 276 | N | H |
| 293 | E | D |
| 316 | T | A |
| 319 | Q | R |
| 340 | S (absent) | S |
| 341 | E (absent) | E |
| 342 | S (absent) | S |

### Example 10. Filaggrin Consensus Sequence

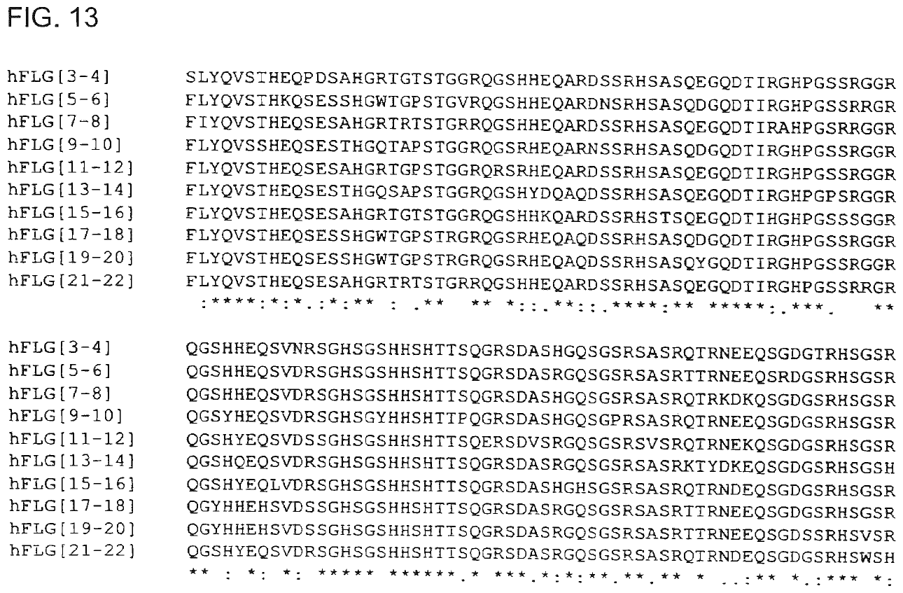

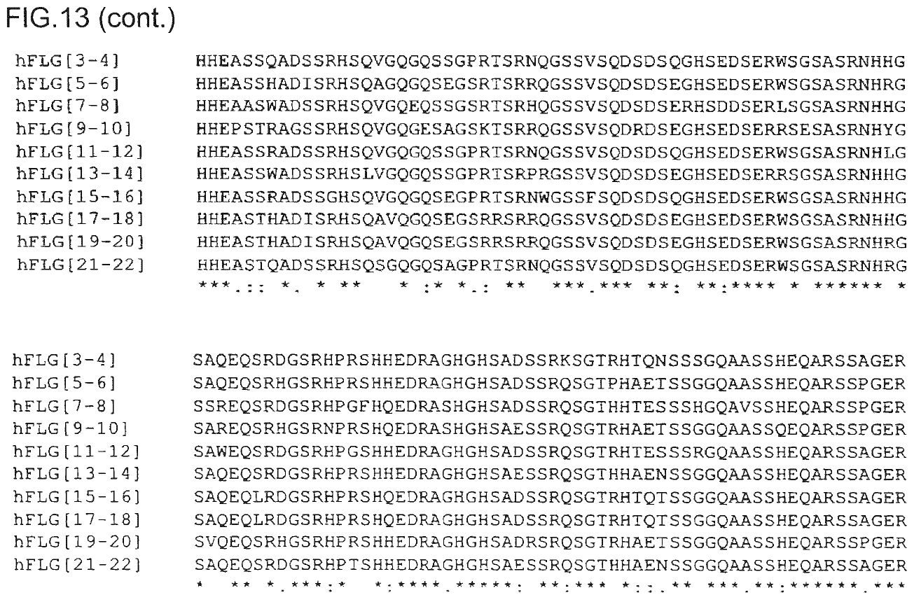

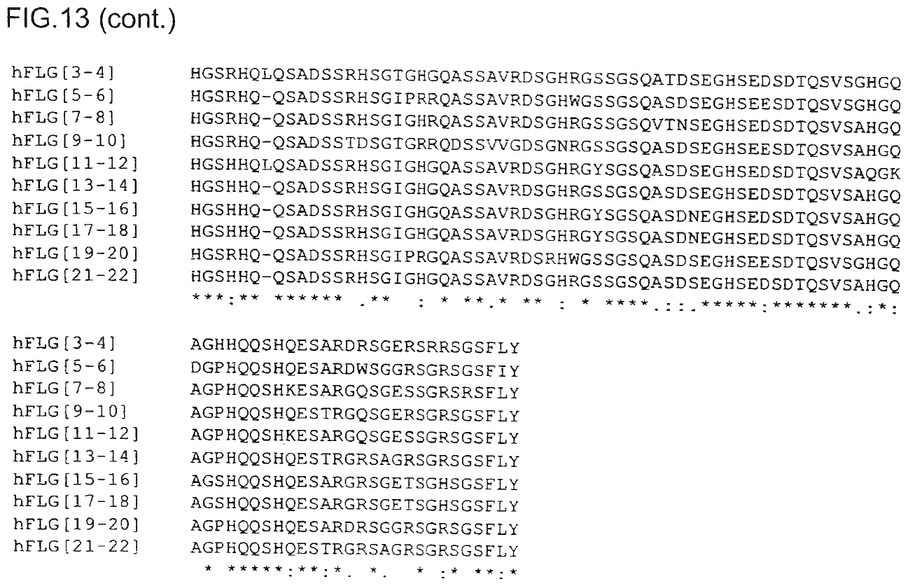

A human filaggrin consensus sequence was generated by alignment of filaggrin dimers hFLG[3-4], hFLG[5-6], hFLG[7-8], hFLG[9-10], hFLG[11-12], hFLG[13-14], hFLG[15-16], hFLG[17-18], hFLG[19-20], hFLG[21-22] as shown in FIG. 13. The consensus sequence shown as SEQ ID NO: 9 refers to the sequence formed from the most frequently occurring amino acids shown in the alignment in FIG. 13.

SEQ ID NO: 9

| | |
|---|---|
| XLYQVSTHXQXDSXHGXTXXSTXXRQXSHXXQAXXXSRHSXSQXGQ | 100 |
| DTIRGHPGXXXXGRQGXXXEXXVXXSGHSGXHHSHTTXQXRSDASH | |
| GXSGXRSA | |
| SRXTXXXXQSXDXTRHSXSRHHEXXSXAXXSXHSXXGQXXSXGXRX | 200 |
| SRXXGSSXSQDXDSXXHSEDSERXSXSASRNHXGSXXEQXRXGSRX | |
| PXXHXEDR | |
| AXHGHSADXSRKSGTXHXXXSSXGQAASSXEQARSSXGERHGSRHQ | 300 |
| XQSADSSXXSGXXHXQXSSAVXDSXXXGXSGSQATXXEGHSEDSDT | |
| QSVSGXGX | |
| XGXHQQSHXESXRXXSGXXSXRSXSFLY | 328 |

### Example 11. Keratin Binding Assay to Measure Activity of Filaggrin

A keratin binding assay was used to measure activity of various hFLG[9-10] sequences set forth in SEQ ID NOs 1-9.

### Keratin Extraction From Human Callus

Keratins were extracted from human callus. One to two grams of callus was homogenized in 10 mL 5 mM Tris pH 7.4 with protease inhibitors using a BULLET BLENDER bead beater. Homogenized tissue was centrifuged 10 000 rpm for 20 minutes at 4° C. The resulting pellet was resuspended in 0.05M tris pH 7.4 containing 8 M urea and 0.025 mM β-mercaptoethanol and mixed gently for 2 h at 37° C. The urea lysate was centrifuged 10 000 rpm for 10 minutes. The supernatant was dialyzed against 5 mM Tris pH7.4 overnight at 4° C. allowing the gradual assembly of keratin filaments.

### Filaggrin—Keratin Binding Assay

An aliquot of assembled callus keratin filaments (above section) was centrifuged 5 minutes at 10 000 RPM to remove insoluble contaminants. Wells were coated overnight at 4° C. with specified amount of keratin extract diluted in coating buffer to a final volume of 200 μL. Coating buffer was removed, wells were blocked with 200 μl 5% non-fat dry milk in PBST (PBS+0.05% Tween20) and incubated at 37° C. for 2 h. Blocking buffer was removed and a pre-determined amount (μg) of the appropriate human recombinant filaggrin protein, diluted in PBST to a final volume of 200 μL, was added and plates were incubated at 37° C. for 2 h. Plates were washed 3 times with 200 μL per well of PBST. For the detection of filaggrin binding, an anti-filaggrin IgY chicken antibody (RL-012-001B antibody 1/1000 in PBST) was added to the wells. For the detection of keratin (controls) 200 μL of a mouse pan-keratin antibody (Type II AE3 Ab at a dilution of 1/500 in PBST) was added to indicated wells. Primary antibodies were incubated at room temperature with shaking for 1 h. Primary antibodies were removed and plates washed 3 times with PBST. To all the wells containing the anti-chicken antibody a secondary alpaca anti-chicken antibody conjugated with horse radish peroxidase (HRP) was added at a dilution of 1/2000 in PBST. Wells containing the anti-keratin antibody received a goat anti-mouse antibody conjugated with HRP at a dilution of 1/500 in PBST. Plates were incubated at room temperature with shaking for 1 h. Plates were washed 3 times with PBST and 100 ul of 1-Step Ultra TMB substrate was added. Plates were incubated at room temperature until the appearance of blue color subsided, 15-20 minutes. The reaction was stopped by adding 100 μL of 2M HCL and absorbances were read on a spectrophotometer at 450-550 nm.

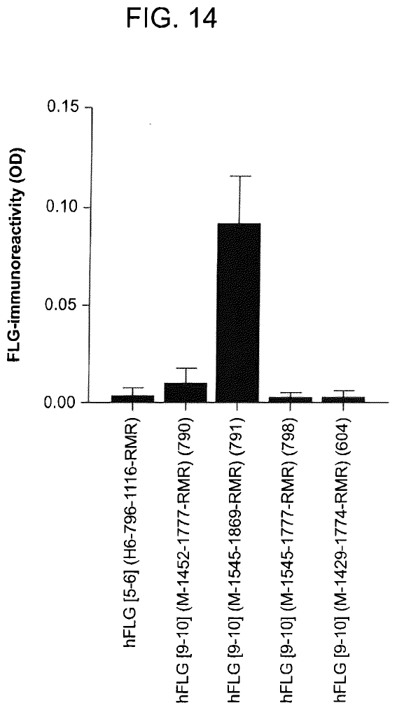

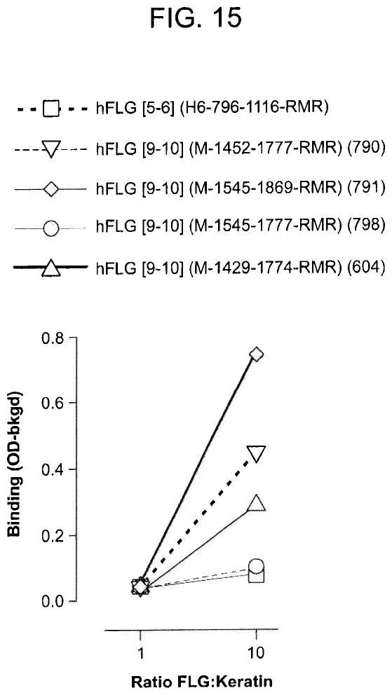

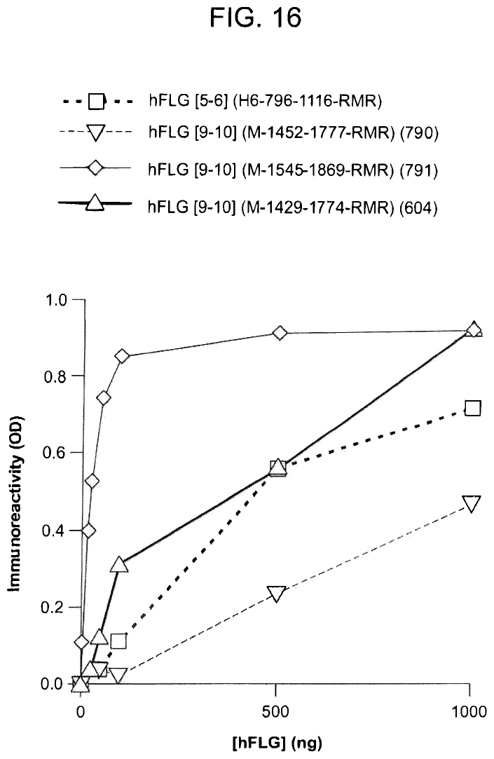

The results are shown in FIGS. 14-16, and demonstrate that there was differential binding to keratin among the hFLG sequences tested. FIG. 14 is a graph that shows background FLG binding at 1 μg/well for 2 h at 37° C. The results shown in FIG. 14 include non specific binding (NSB). FIG. 15 is a graph that shows the binding of various hFLG segments to human callus keratin (with NSB removed). FIG. 16 is a graph that shows titration of IgY anti-hFLG. As shown in FIGS. 14-16, hFLG[5-6] did not bind keratin, while the various hFLG[9-10] sequences that were tested showed binding to keratin.

### Example 12. Activity of hFLG[9-10]-Secreting SE in Mice

A genetic IV mouse model (Flg−/−) will be used, as well as wild type mice to assess colonization dynamics of FLG-producing SE in vivo. Flg−/− mice are filaggrin deficient and exhibit dry, scaly skin. Despite marked decreases in natural moisturizing factor levels, which are filaggrin degradation products, stratum corneum (SC) hydration and TEWL are normal in Flg−/− mice. Antigens penetrate the Flg−/− SC more efficiently, leading to enhanced responses in hapten-induced contact hypersensitivity and higher serum levels of anti-ovalbumin (OVA) IgG(1) and IgE. Flg−/− mice are obtained from RIKEN BioResource Research Center (RIKEN BRC, Tsukuba, Ibaraki, Japan). Wild type mice (BALB/c) will also be used in this experiment.

The hFLG[9-10] sequences set forth in SEQ ID NOs 1-9 will be used in SE to Flg−/− and BALB/c mice. The study will be conducted for four weeks using five groups in each mouse type. Mice will be assigned into the following treatment groups: topical vehicle control (50% glycerol, 50% sterilized BHI medium), topical wild type SE (1.0×10⁸ CFU/cm² in 50% glycerol), and three doses of each of each filaggrin-secreting SE constructs (SE^{FLG}) (10⁶, 10⁷, and 10⁸ CFU/cm² in 50% glycerol). Each solution will be applied to the same ear and tail on each mouse daily for seven days, and mice be assessed on days 7, 14, 30, and 60 for microbiome analyses to assess colonization dynamics and on days 7 and 14 for microscopy and histology to assess localization and macroscopic changes in the skin (e.g., any signs of adverse events such as inflammation), etc. 12 mice in each arm per mouse type will be used.

### Equivalents

Those skilled in the art will recognize, or be able to ascertain using no more than routine experimentation, many equivalents to the specific embodiments of the invention described herein. Such equivalents are intended to be encompassed by the following claims.

1. A recombinant bacterium, or combination of bacteria, capable of secreting a filaggrin (FLG) polypeptide, wherein the recombinant bacterium, or combination of bacteria comprises an expression vector comprising a first coding sequence comprising a gene capable of expressing the FLG polypeptide and a second coding sequence comprising a gene capable of expressing a cell penetrating peptide.

2. The recombinant bacterium, or combination of bacteria of claim 1, further comprising a third coding sequence comprising a gene capable of expressing an export signal.

3. (canceled)

4. (canceled)

5. (canceled)

6. (canceled)

7. (canceled)

8. The recombinant bacterium, or combination of bacteria of claim 1, wherein the filaggrin (FLG) polypeptide comprises an amino acid sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

9. The recombinant bacterium, or combination of bacteria, of claim 1, wherein the bacterium, or combination of bacteria is selected from the group consisting of Bifidobacterium, Brevibacterium, Propionibacterium, Lactococcus, Streptococcus, Staphylococcus, Lactobacillus, Enterococcus, Pediococcus, Leuconostoc, or Oenococcus, or combinations thereof.

10. (canceled)

11. The recombinant bacterium, or combination of bacteria, of claim 1, wherein the bacterium secretes a filaggrin fusion protein.

12. (canceled)

13. (canceled)

14. (canceled)

15. (canceled)

16. (canceled)

17. (canceled)

18. (canceled)

19. (canceled)

20. (canceled)

21. (canceled)

22. A live biotherapeutic composition comprising a bacterium, or combination of bacteria, wherein the bacterium comprises
(i) a first coding sequence comprising a nucleic acid sequence encoding a filaggrin (FLG) therapeutic polypeptide;
(ii) a second coding sequence comprising a nucleic acid sequence encoding a cell penetrating peptide;
(iii) a third coding sequence comprising a nucleic acid sequence encoding an export signal; and
(iiv) a promoter operably linked to the first coding sequence, the second coding sequence and the third coding sequence;
wherein the first coding sequence, second coding sequence and third coding sequence are capable of expressing a filaggrin fusion product, or variant thereof.

23. The composition of claim 22, wherein the recombinant microorganism is Staphylococcus epidermidis.

24. The composition of claim 22, wherein the export signal exports the filaggrin fusion product, or variant thereof, out of the recombinant microorganism.

25. The composition of claim 22, wherein the cell penetrating peptide facilitates the entry of the filaggrin fusion product, or variant thereof, into a human keratinocyte.

26. (canceled)

27. (canceled)

28. A method of treating a skin disease comprising administering to a subject in need thereof the composition of claim 22.

29. The method of claim 28, wherein the skin disease is atopic dermatitis.

30. The method of claim 28, wherein the composition is applied topically.

31. The composition of claim 22, wherein the filaggrin (FLG) therapeutic polypeptide comprises an amino acid sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

32. The composition of claim 31, wherein the filaggrin (FLG) therapeutic polypeptide comprises an amino acid sequence that is at least 95% identical to a sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

33. The composition of claim 32, wherein the filaggrin (FLG) therapeutic polypeptide comprises an amino acid sequence that is at least 97% identical to a sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

34. The composition of claim 32, wherein the filaggrin (FLG) therapeutic polypeptide comprises an amino acid sequence that is at least 99% identical to a sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

35. The recombinant bacterium, or combination of bacteria of claim 9, wherein the filaggrin (FLG) polypeptide comprises an amino acid sequence at least 95% identical to a sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

36. The recombinant bacterium, or combination of bacteria of claim 35, wherein the filaggrin (FLG) polypeptide comprises an amino acid sequence at least 97% identical to a sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.

37. The recombinant bacterium, or combination of bacteria of claim 36, wherein the filaggrin (FLG) polypeptide comprises an amino acid sequence at least 95% identical to a sequence selected from the group consisting of: SEQ ID NO: 5, SEQ ID NO: 1, SEQ ID NO: 2, SEQ ID NO: 3, SEQ ID NO: 4, SEQ ID NO: 6, SEQ ID NO: 7, SEQ ID NO: 8 and SEQ ID NO: 9.


SEQUENCE LISTING (52 sequences)

SEQ ID NO: 1 - protein, 4061 aa, Homo sapiens
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKEKNKTENTRLGDNRKRLSERLEEKEDNEEGVYDYENTGRMTQKWIQSGHIATYYTIQDEAYDTTDSLLEENKIYERSRSSDGKSSSQVNRSRHENTSQVPLQESRTRKRRGSRVSQDRDSEGHSEDSERHSGSASRNHHGSAWEQSRDGSRHPRSHDEDRASHGHSADSSRQSGTRHAETSSRGQTASSHEQARSSPGERHGSGHQQSADSSRHSATGRGQASSAVSDRGHRGSSGSQASDSEGHSENSDTQSVSGHGKAGLRQQSHQESTRGRSGERSGRSGSSLYQVSTHEQPDSAHGRTGTSTGGRQGSHHEQARDSSRHSASQEGQDTIRGHPGSSRGGRQGSHHEQSVNRSGHSGSHHSHTTSQGRSDASHGQSGSRSASRQTRNEEQSGDGTRHSGSRHHEASSQADSSRHSQVGQGQSSGPRTSRNQGSSVSQDSDSQGHSEDSERWSGSASRNHHGSAQEQSRDGSRHPRSHHEDRAGHGHSADSSRKSGTRHTQNSSSGQAASSHEQARSSAGERHGSRHQLQSADSSRHSGTGHGQASSAVRDSGHRGSSGSQATDSEGHSEDSDTQSVSGHGQAGHHQQSHQESARDRSGERSRRSGSFLYQVSTHKQSESSHGWTGPSTGVRQGSHHEQARDNSRHSASQDGQDTIRGHPGSSRRGRQGSHHEQSVDRSGHSGSHHSHTTSQGRSDASRGQSGSRSASRTTRNEEQSRDGSRHSGSRHHEASSHADISRHSQAGQGQSEGSRTSRRQGSSVSQDSDSEGHSEDSERWSGSASRNHRGSAQEQSRHGSRHPRSHHEDRAGHGHSADSSRQSGTPHAETSSGGQAASSHEQARSSPGERHGSRHQQSADSSRHSGIPRRQASSAVRDSGHWGSSGSQASDSEGHSEESDTQSVSGHGQDGPHQQSHQESARDWSGGRSGRSGSFIYQVSTHEQSESAHGRTRTSTGRRQGSHHEQARDSSRHSASQEGQDTIRAHPGSRRGGRQGSHHEQSVDRSGHSGSHHSHTTSQGRSDASHGQSGSRSASRQTRKDKQSGDGSRHSGSRHHEAASWADSSRHSQVGQEQSSGSRTSRHQGSSVSQDSDSERHSDDSERLSGSASRNHHGSSREQSRDGSRHPGFHQEDRASHGHSADSSRQSGTHHTESSSHGQAVSSHEQARSSPGERHGSRHQQSADSSRHSGIGHRQASSAVRDSGHRGSSGSQVTNSEGHSEDSDTQSVSAHGQAGPHQQSHKESARGQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRNEKQSGDGSRHSGSRHHEASSRADSSRHSQVGQGQSSGPRTSRNQGSSVSQDSDSQGHSEDSERWSGSASRNHLGSAWEQSRDGSRHPGSHHEDRAGHGHSADSSRQSGTRHTESSSRGQAASSHEQARSSAGERHGSHHQLQSADSSRHSGIGHGQASSAVRDSGHRGYSGSQASDSEGHSEDSDTQSVSAQGKAGPHQQSHKESARGQSGESSGRSGSFLYQVSTHEQSESTHGQSAPSTGGRQGSHYDQAQDSSRHSASQEGQDTIRGHPGPSRGGRQGSHQEQSVDRSGHSGSHHSHTTSQGRSDASRGQSGSRSASRKTYDKEQSGDGSRHSGSHHHEASSWADSSRHSLVGQGQSSGPRTSRPRGSSVSQDSDSEGHSEDSERRSGSASRNHHGSAQEQSRDGSRHPRSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRTGTSTGGRQGSHHKQARDSSRHSTSQEGQDTIHGHPGSSSGGRQGSHYEQLVDRSGHSGSHHSHTTSQGRSDASHGHSGSRSASRQTRNDEQSGDGSRHSGSRHHEASSRADSSGHSQVGQGQSEGPRTSRNWGSSFSQDSDSQGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQYGQDTIRGHPGSSRGGRQGYHHEHSVDSSGHSGSHHSHTTSQGRSDASRGQSGSRSASRTTRNEEQSGDSSRHSVSRHHEASTHADISRHSQAVQGQSEGSRRSRRQGSSVSQDSDSEGHSEDSERWSGSASRNHRGSVQEQSRHGSRHPRSHHEDRAGHGHSADRSRQSGTRHAETSSGGQAASSHEQARSSPGERHGSRHQQSADSSRHSGIPRGQASSAVRDSRHWGSSGSQASDSEGHSEESDTQSVSGHGQAGPHQQSHQESARDRSGGRSGRSGSFLYQVSTHEQSESAHGRTRTSTGRRQGSHHEQARDSSRHSASQEGQDTIRGHPGSSRRGRQGSHYEQSVDRSGHSGSHHSHTTSQGRSDASRGQSGSRSASRQTRNDEQSGDGSRHSWSHHHEASTQADSSRHSQSGQGQSAGPRTSRNQGSSVSQDSDSQGHSEDSERWSGSASRNHRGSAQEQSRDGSRHPTSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLYQVSTHEQSESAHGRAGPSTGGRQGSRHEQARDSSRHSASQEGQDTIRGHPGSRRGGRQGSYHEQSVDRSGHSGSHHSHTTSQGRSDASHGQSGSRSASRETRNEEQSGDGSRHSGSRHHEASTQADSSRHSQSGQGESAGSRRSRRQGSSVSQDSDSEAYPEDSERRSESASRNHHGSSREQSRDGSRHPGSSHRDTASHVQSSPVQSDSSTAKEHGHFSSLSQDSAYHSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 2 - protein, 346 aa, synthetic construct
  REGION 1..346: note = Description of Artificial Sequence: Synthetic polypeptide
MQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQ

SEQ ID NO: 3 - protein, 356 aa, synthetic construct
  REGION 1..356: note = Description of Artificial Sequence: Synthetic polypeptide
MQSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQRMRRMRRMRR

SEQ ID NO: 4 - protein, 337 aa, synthetic construct
  REGION 1..337: note = Description of Artificial Sequence: Synthetic polypeptide
MESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESRMRRMRRMRR

SEQ ID NO: 5 - protein, 336 aa, synthetic construct
  REGION 1..336: note = Description of Artificial Sequence: Synthetic polypeptide
MRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRRMRRMRRMRR

SEQ ID NO: 6 - protein, 244 aa, synthetic construct
  REGION 1..244: note = Description of Artificial Sequence: Synthetic polypeptide
MRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQSESRMRRMRRMRR

SEQ ID NO: 7 - skipped

SEQ ID NO: 8 - protein, 352 aa, synthetic construct
  REGION 1..352: note = Description of Artificial Sequence: Synthetic polypeptide
MRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEASTRADSSRHSQVGQGQSSGSRTSRRQGSSVSQDSDSEGHSEDSERRSGSASRNHYGSAQEQSRDGSRHPRSHQEDRASHGHSAESSRQSGTRHAETSSGGQAASSHEQARSSPGERHGSRHQQSADSSRHSGIGHGQASSAVRDSGHRGSSGSQASDSEGHSEDSDTQSVSAHGQAGPHQQSHQESARGRSGERSGRSGSFLYQVSTHEQSESRMRRMRRMRR

SEQ ID NO: 9 - protein, 328 aa, synthetic construct
  REGION 1..328: note = Description of Artificial Sequence: Synthetic polypeptide
  VARIANT 1: note = MOD_RES - Any amino acid
  VARIANT 9: note = MOD_RES - Any amino acid
  VARIANT 11: note = MOD_RES - Any amino acid
  VARIANT 14: note = MOD_RES - Any amino acid
  VARIANT 17: note = MOD_RES - Any amino acid
  VARIANT 19..20: note = MOD_RES - Any amino acid
  VARIANT 23..24: note = MOD_RES - Any amino acid
  VARIANT 27: note = MOD_RES - Any amino acid
  VARIANT 30..31: note = MOD_RES - Any amino acid
  VARIANT 34..36: note = MOD_RES - Any amino acid
  VARIANT 41: note = MOD_RES - Any amino acid
  VARIANT 44: note = MOD_RES - Any amino acid
  VARIANT 55..58: note = MOD_RES - Any amino acid
  VARIANT 63..65: note = MOD_RES - Any amino acid
  VARIANT 67..68: note = MOD_RES - Any amino acid
  VARIANT 70..71: note = MOD_RES - Any amino acid
  VARIANT 77: note = MOD_RES - Any amino acid
  VARIANT 84: note = MOD_RES - Any amino acid
  VARIANT 86: note = MOD_RES - Any amino acid
  VARIANT 94: note = MOD_RES - Any amino acid
  VARIANT 97: note = MOD_RES - Any amino acid
  VARIANT 103: note = MOD_RES - Any amino acid
  VARIANT 105..108: note = MOD_RES - Any amino acid
  VARIANT 111: note = MOD_RES - Any amino acid
  VARIANT 113: note = MOD_RES - Any amino acid
  VARIANT 118: note = MOD_RES - Any amino acid
  VARIANT 124..125: note = MOD_RES - Any amino acid
  VARIANT 127: note = MOD_RES - Any amino acid
  VARIANT 129..130: note = MOD_RES - Any amino acid
  VARIANT 132: note = MOD_RES - Any amino acid
  VARIANT 135..136: note = MOD_RES - Any amino acid
  VARIANT 139..140: note = MOD_RES - Any amino acid
  VARIANT 142: note = MOD_RES - Any amino acid
  VARIANT 144: note = MOD_RES - Any amino acid
  VARIANT 146: note = MOD_RES - Any amino acid
  VARIANT 149..150: note = MOD_RES - Any amino acid
  VARIANT 154: note = MOD_RES - Any amino acid
  VARIANT 158: note = MOD_RES - Any amino acid
  VARIANT 161..162: note = MOD_RES - Any amino acid
  VARIANT 170: note = MOD_RES - Any amino acid
  VARIANT 172: note = MOD_RES - Any amino acid
  VARIANT 179: note = MOD_RES - Any amino acid
  VARIANT 182..183: note = MOD_RES - Any amino acid
  VARIANT 186: note = MOD_RES - Any amino acid
  VARIANT 188: note = MOD_RES - Any amino acid
  VARIANT 192: note = MOD_RES - Any amino acid
  VARIANT 194..195: note = MOD_RES - Any amino acid
  VARIANT 197: note = MOD_RES - Any amino acid
  VARIANT 202: note = MOD_RES - Any amino acid
  VARIANT 209: note = MOD_RES - Any amino acid
  VARIANT 216: note = MOD_RES - Any amino acid
  VARIANT 218..220: note = MOD_RES - Any amino acid
  VARIANT 223: note = MOD_RES - Any amino acid
  VARIANT 230: note = MOD_RES - Any amino acid
  VARIANT 237: note = MOD_RES - Any amino acid
  VARIANT 247: note = MOD_RES - Any amino acid
  VARIANT 254..255: note = MOD_RES - Any amino acid
  VARIANT 258..259: note = MOD_RES - Any amino acid
  VARIANT 261: note = MOD_RES - Any amino acid
  VARIANT 263: note = MOD_RES - Any amino acid
  VARIANT 268: note = MOD_RES - Any amino acid
  VARIANT 271..273: note = MOD_RES - Any amino acid
  VARIANT 275: note = MOD_RES - Any amino acid
  VARIANT 282..283: note = MOD_RES - Any amino acid
  VARIANT 298: note = MOD_RES - Any amino acid
  VARIANT 300..301: note = MOD_RES - Any amino acid
  VARIANT 303: note = MOD_RES - Any amino acid
  VARIANT 309: note = MOD_RES - Any amino acid
  VARIANT 312: note = MOD_RES - Any amino acid
  VARIANT 314..315: note = MOD_RES - Any amino acid
  VARIANT 318..319: note = MOD_RES - Any amino acid
  VARIANT 321: note = MOD_RES - Any amino acid
  VARIANT 324: note = MOD_RES - Any amino acid
XLYQVSTHXQXDSXHGXTXXSTXXRQXSHXXQAXXXSRHSXSQXGQDTIRGHPGXXXXGRQGXXXEXXVXXSGHSGXHHSHTTXQXRSDASHGXSGXRSASRXTXXXXQSXDXTRHSXSRHHEXXSXAXXSXHSXXGQXXSXGXRXSRXXGSSXSQDXDSXXHSEDSERXSXSASRNHXGSXXEQXRXGSRXPXXHXEDRAXHGHSADXSRKSGTXHXXXSSXGQAASSXEQARSSXGERHGSRHQXQSADSSXXSGXXHXQXSSAVXDSXXXGXSGSQATXXEGHSEDSDTQSVSGXGXXGXHQQSHXESXRXXSGXXSXRSXSFLY

SEQ ID NO: 10 - protein, 416 aa, Homo sapiens
AGPHQQSHQESTRGRSAGRSGRSGSFLYQVSTHEQSESAHGRTGTSTGGRQGSHHQQARDSSRHSTSQEGQDTIHGHRGSSSGGRQGSHYEQLVDRSGHSGSHHSHTTSQGRSDASHGHSGSRSASRQTRNDEQSGDGSRHSGSRHHEASSRADSSGHSQVGQGQSEGPRTSRNWGSSFSQDSDSQGHSEDSERWSGSASRNHHGSAQEQLRDGSRHPRSHQEDRAGHGHSADSSRQSGTRHTQTSSGGQAASSHEQARSSAGDRHGSHHQQSADSSRHSGIGHGQASSAVRDSGHRGYSGSQASDNEGHSEDSDTQSVSAHGQAGSHQQSHQESARGRSGETSGHSGSFLYQVSTHEQSESSHGWTGPSTRGRQGSRHEQAQDSSRHSASQDGQDTIRGHPGSSRGGRQGYHHEQ

SEQ ID NO: 11 - protein, 4061 aa, Homo sapiens; identical to SEQ ID NO: 1

SEQ ID NO: 12 - protein, 4384 aa, Mus musculus
  VARIANT 1045: note = MOD_RES - Any amino acid
  VARIANT 1048: note = MOD_RES - Any amino acid
MSALLESITSMIEIFQQYSTSDKEEETLSKEELKELLEGQLQAVLKNPDDQDIAEVFMQMLDVDHDDKLDFAEYLLLVLKLAKAYYEASKNESFQTHGSNGRSKTDYKGLEEEGEEGNKQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSSHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSRYYYEQEHSEEESDSQHQHGHQHEQQRGHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGTSAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGNQGAHQKQGRDSARSRGSNQGHSSSRHQADSPRVSARSGSXGRXQSPDASGRNSTKRDRPRQPSPSQSSDSHVHSGKAGSISGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSRYYYEQEHSEEESDSQHQHGHQHEQQRGHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRIGSSADRQGRRGVSESQASDSEGHSDFSEGQAVGAHRQSEAGQRHEQRSSRGQHGSGYYYEQEHSEEESDSQHQHGHQHEQQRGTQHQHEHQQPESGHRQQQSSGRGHQGTHQEQGRDSARSRGSNQGHSSSRHQADSPRVSARSGSGGRGQSPDASGRSSNRRDRPRQPSPSQSSDSQVHSGVQVEGRRRQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSGSQASDSEGHSDFSEGQAVGAHRQSGAGQRHEQRSSRGQHGSGYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRSQHGSGYYYEQEHSEEESDSQHQHSHQHEQQRGHQHQHQHQHEHEQPESGHRQQQSSGRGNQGAHQEQGRDSARSRGSNQGHSSSRHQADSPRVSARSGSGGRGQSPDASGRSSNRRDRPRQPSPSQSSDSHVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRSRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSGSQASDSEGHSDFSEGQAVGAHRQSGAGQRHEQRSSRGQHGSGYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQQGHQHEQQRGHQHQHQHQHEHEQPESGHRQQQSSGRGHQGAHQEQGRDSARSRGSNQGHSSSRHQADSPRVSARSGSGGRGQSPDGSGRSSNRRDRPRQPSASQSSDSQVHSGVQVEAQRGQSSSANRRAGSSSGSGVQSAAASGQGGYESIFTAKHLDFNQSHSYYYY

SEQ ID NO: 13 - protein, 554 aa, Mus musculus
MSALLESITSMIEIFQQYSTSDKEEETLSKEELKELLEGQLQAVLKNPDDQDIAEVFMQMLDVDHDDKLDFAEYLLLVLKLAKAYYEASKNESFQTHGSNGRSKTDYKGLEEEGEEGNEQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSFHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGSGVQGASAGGLAADASRRTGALQGQASAQGRAGSQGQAQGRVGSSADRQGRRGVSESQASDSEGQSDFSEGQAVGAHRQSGAGQRHEQRSSRGQYGSGFYPVYYYYEQEHSEEESDSQHGHQHEQQRGHQHQHQHEHEQPESGHRQQQSSGRGHQGAHQEQGRDSARSRGSNQGHSSSRHQADSPRVSVRSGSGGRGQ

SEQ ID NO: 14 - protein, 336 aa, Mus musculus
PDGSGRSSNRRDRPRQLSPSQSSDSQVHSGVQVEGRRGHSSSANRRAGSSSGSGVQGASAGGLAADASRRSGARQGQASAQGRAGSQGQAQGRVSSSADRQGRRGVSESRASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARPRGSNQGHSSSRHQADSPRVSARSGSGGRGQSPDASGRSSNRRDRPRQPSPSQSSDSQVHSGVQVEAQRGQSSSANRRAGSSSGSGVQGAAASGQGGYESIFTAKHLDFNQSHSYYYY

SEQ ID NO: 15 - protein, 250 aa, Mus musculus
GGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASA

SEQ ID NO: 16 - protein, 2642 aa, Mus musculus
MSTNSPSPATGSSSSADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQHSHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRSQHGSGYYYEQEHSEEESDSQHQHSHQHEQQRGHQHQHQHQHEHEQPESGHRQQQSSGRGNQGAHQEQGRDSARSRGSNQGHSSSRHQADSPRVSARSGSGGRGQSPDASGRSSNRRDRPRQPSPSQSSDSHVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRSRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGALQGQASAQGRAGSQGQAQGRVGSSADRQGRRGVSESQASDSEGHSDFSEGQAVGAHRQSGAGQRHEQRSSRGQHGSGYYYEQEHSEEESDSQHQHGHQHEQQRGHQHQHQHQHEHEQPESGHRQQQSSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSGSQASDSEGHSDFSEGQAVGAHRQSGAGQRHEQRSSRGQHGSGYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSSSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGFYPVYYYYEQEHSEEESDSQHQHGHQHEQQRGHQHQHQHQHEHEQPESGHRQQQFSGRGHQGAHQEQGRDSARSRGSNQGHSSSRHQADSPRVSARSGSGGRGQSPDASGRSSNRRDRPRQPSPSQSSDSQVHSGVQVEGRRGQSSSANRRAGSSSGSGVQGASAGGLAADASRRSGARQGQASAQGRAGSQGQAQGRVGSSADRQGRRGVSESQASDSEGHSDFSEGQAVGAHRQSGAGQRHEQRSSRGQHGSGYYYEQEHSEEESDSQHQQGHQHEQQRGHQHQHQHQHEHEQPESGHRQQQSSGRGHQGAHQEQGRDSARSRGSNQGHSSSRHQADSPRVSARSGSGGRGQSPDGSGRSSNRRDRPRQPSASQSSDSQVHSGVQVEAQRGQSSSANRRAGSSSGSGVQSAAASGQGGYESIFTAKHLDFNQSHSYYYY

SEQ ID NO: 17 - protein, 336 aa, Mus musculus; identical to SEQ ID NO: 14

SEQ ID NO: 18 - protein, 357 aa, Mus musculus
MSALLESITSMIEIFQQYSTSDKEEETLSKEELKELLEGQLQAVLKNPDDQDIAEVFMQMLDVDHDDKLDFAEYLLLVLKLAKAYYEASKNESFQTHGSNGRSKTDYKGLEEEGEEGNKQNLRRRHGGTDGKRKSDRTRSPNGKRGKRQESRCRSEGKDKHRREPEKHRHQQDSKRKQRHGSGSTERKDNRNKKNRQSKERNYDEIYDNGKYNEDWEASYNNCYYKTQNTTLDQREGNRRPRADSQKEPQSSHGQADNSDSEGGRQQSHSKPSPVRADQRRSRAGQAGSSKVSARSGSGGRGQSPDGSGRSSNRRDRPRQPSPSQSSDSQVHSGVQVEGRRGQSSSANRRAGSSSGS

SEQ ID NO: 19 - protein, 366 aa, Rattus norvegicus
MSTLLESITSMIDIFQQYSNNDKEEETLSKEELKELLEGELQAVLKNPNDQDIAEVFMQMLDVDHDDKIDFTEYLLMVLKLAQAYYETSKKRRSQTKESGKRNEHDYKGYEERREKVQRRHRRRNSGTDGKQENERSKSPRGRGNKRRGSSTISEESDTNRNSDTENKRHHHGSNRRQRRGSNSSDRKETRSKKHREVKERNAGIYNDGKDGQDWEVNYENCYYKTEESNREQREGRNHKTKESHSESEASGGQAGRRGTAATRHTSRPEQSPDTAGRTGSSRGQQSAQRHADSTPGSTRTGSRGRGESPAGQQSPDRARHIESRRGRTREASASQSSDSEGHSGAHAGIGQGQTSTTHRRAGSSS

SEQ ID NO: 20 - protein, 3088 aa, Pan troglodytes
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKQKNKTENTRLEDNRKRLSERLEEKEDNEEGGYDYENTGRMTQKWIQSGHIATYYTIQDEAYDTTDNLLEENKIYERSRSSDDKSSSQVNRSRHENTSQVPLQESRTRKRRGSRVSQDRDSEGHSEDSERHSGSASRNHPGSARQQSRDGSRHPRSHDEDRASHGHSADSSRQSGTRHAETSSRGQTASSHEQARSSPGERHGSRHQQSADSSRHSATGRGQASSAVSDRGPRGSSGSQASDSEGHSENSDTQSVSGHGKAGPRQQSHQESARGRSGERSGHSGSFLYQVSTHEQSDSAHGRTGTSTGGRQGSHHEQARDSSRHSASQEGQDTIRGHPGSSRGGRQGSHHEQLVNRSGHSGSHHSHTTSQGRSDASHGQSGSRSASRQTRNEEQSGDGSRHSGSRHHEASSQADSSRHSQVGQGQSSGPRTSSHQGSSVSQDSDSEGHSEDSERWSGSASRNHHGSAREQSRDGSRHPRSHHEDRAGHGHSADSSRQSGTRHTESSSRGQAVSSHEQARSSPGERHGSRHQQSADSSRHSGIGHGQASSAVRDSGHRGSSGSQAIDSEGHSEDSDTQSVSSHGQAGPHQQSHKESARGQSGESSGRSGSFLYQVSSHEQSESTYGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRASSSRHSQVGQGESAVSKTSRRQGSSVSQDRDSEGHSEDSERQSESASRNHYGSAREQSRHGSRNPRSHQEDRASHGHSAESSRQSGTHHAETSSHGQAASSQEQARSSRGERHGSRHQQSADSSTDSGTGGRQASSVVGDSGNRGSSGSQASDSEGHSEDSDTQSVSAHGQAGPRQQSHQESTRGQSGERSGRSGSFLYQVSTHEQSESAHGRTGPSTGGRQRSRHEQARDSSRHSASQESQDIIHAHPGSSRGGRQGSHYEQSVDRSGHSGSHHSHTTSQERSNASHGQSGSRSASRQTRNEEQSGDGSRHSGSRHHEASSRADSSRHSQVGQGQSSGPRTSRNQGSSVSQDSDSQGHSEDSERWSGSASRNHHGSAWEQSRDGSRHPGSHQEDRAGHGHSADSSRQSGTHHTESSSRGQAASSHEHARSSAGERHGSHHQQSADSSRHSGIGHGQASSAVRDSGHRGSSGSQASDSEGHSEDSDTQSLSAHGQAGPHQQSHQESTRGRSAERSGRSGSFLYQVSTHEQSESAHGRTGTSTGGRKRSLHEQARDSSRHSVSQEGQDTIHGHAGSSSGGRQGSHYEQLVDRSGHSGSHHSHTTSQGRSDAYHGQSGSRSASRQTHNDEQSGDGFRHSGSHHHEASSRADSSRHSQVGQGQSEGPRTSRHRESSVSQDSDSEGHSEDSERWSGSASRNHHGSAREQSRDGSRHPRSHHEDRAGHGHSADSSRQSGTRHTQTSSGGQAASSHEQGRSSAGERHGSRHQQSADSSRHSGIGHGQASSAVRDSGHRGYSGSQASDSEGHSEDSDTQSVSAHGQAGSHQQSHQESARGRSGERSGRSGSFLYQVSTHEQSESSHGWTGPSTRGRQGSRHEQAQDSSRHSASQEGQDTIRGHPGPSRGGRQGYHHEQSVDSSGHSGSHHSHITSQGSSHASHGQSGSRSASRTTRNDEQSVDGSRHSGSRHHEASTHADISRHSQAGQGQSEGSRTSRRQGSSVSQDSDSEGHSEDSERWSGSASRNHRGSAQEQSRDGSRHPRSHHEDRAGHGHSADSSRQSGTHHAETSSGGQAASSREQARSSPGERHGSRHQQSADSSRHSGIRRGQASSAVRDSGHWGSSGSQASDSEGHSEESDTQSVSGHGQDGPHQQSHQESARDRSGGRSGRSGSFLYQVSTHEQSESTHGQTGTSTGGRQGSHHEQARDSSRHSASQEGQDTIHAHPGSSRGGRQGSHHEQSVDTSGHSGSHHSHTTSQGRSDASHGQSGSRSASRQTRNDEQSGDGSRHSGSHHHEAFTQADSSRHSQSGQGESAGSRRSRRQGSSVSQDSDSQGHSEDSERWSGSASRNQHGSAREQSRDGSRHPGSHQEDRAGHGHSADSSRQSGTRHTESSSRGQAASSHEQARSSAGERHGSRHQLQSADSSRHSGIGHGQASSAVRDSGHRGSSGSQAIDSEGHSEDSDTQSVSAQGQAGPHQRSHKESARGQSGESSGRSGSFLYQVSTHEQPESTHGQSAPSTGGRQGSHHDQAQDSSRHSASQEGQDTIRGHPGPSRGGRQGSHHKQSVDRSGHSGSHHSHTTSQGRSDASRGQSGSRSASRQTHDKEQSGDGSRHSGSRHHEASSWADSSRHSQAGQGQSEGSRTSRRQGSSFSQDSDSEGHSEDSERRSGSASRNHRGSAQEQSRDGSRHPRSHHEDRAGHGHSADSSRQSGTHHAQNSSGGQAASFHEQARSSAGERHGSHHQQSADSSRHSGIGHGQASSAVRDSGHRGSSGSQASDSEGHSEDSDTQSVSAHGQAGPHQQSHQESTRGRSAERSGRSGSFLYQVSTHEQSESAHGRTGPSTGGRQGSRHEQARDSSRHSASQEGQDTIHGHPGSRRGGRQGSYHEQSVDRSGHSGSHHSHTTSQGRSDASHGQSGSRSASRQTRNEQQSGDGSRHSGSSHHEASTQADSSRHSQSGQGESAGSRRSRRQGSSVSQDSDSEAYPEDSERRSESASRNRHGSSREQSRDGSRHPGSSHRDTTRHVQSSPVQSDSSTAKEHGHFSSLSQDSAYRSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 21 - protein, 2764 aa, Pan troglodytes
MSTLLENIFAIINLFKQYSKKDKNTDTLSKKELKELLEKEFRQILKNPDDPDMVDVFMDHLDIDHNKKIDFTEFLLMVFKLAQAYYESTRKENLPISGHKHRKHSHHDKHEDNKQEENKENRKRPSSLERRNNRKGNKGRSKSPRETGGKRHESSSEKKERKGYSPTHREEEYGKNHHNSSKKQKNKTENTRLEDNRKRLSERLEEKEDNEEGGYDYENTGRMTQKWIQSGHIATYYTIQDEAYDTTDNLLEENKIYERSRSSDDKSSSQVNRSRHENTSQVPLQESRTRKRRGSRVSQDRDSEGHSEDSERHSGSASRNHPGSARQQSRDGSRHPRSHDEDRASHGHSADSSRQSGTRHAETSSRGQTASSHEQARSSPGERHGSRHQQSADSSRHSATGRGQASSAVSDRGPRGSSGSQASDSEGHSENSDTQSVSGHGKAGPRQQSHQESARGRSGERSGHSGSFLYQVSTHEQSDSAHGRTGTSTGGRQGSHHEQARDSSRHSASQEGQDTIRGHPGSSRGGRQGSHHEQLVNRSGHSGSHHSHTTSQGRSDASHGQSGSRSASRQTRNEEQSGDGSRHSGSRHHEASSQADSSRHSQVGQGQSSGPRTSSHQGSSVSQDSDSEGHSEDSERWSGSASRNHHGSAREQSRDGSRHPRSHHEDRAGHGHSADSSRQSGTRHTESSSRGQAVSSHEQARSSPGERHGSRHQQSADSSRHSGIGHGQASSAVRDSGHRGSSGSQAIDSEGHSEDSDTQSVSSHGQAGPHQQSHKESARGQSGESSGRSGSFLYQVSSHEQSESTYGQTAPSTGGRQGSRHEQARNSSRHSASQDGQDTIRGHPGSSRGGRQGSYHEQSVDRSGHSGYHHSHTTPQGRSNASHGQSGSRSASRQTRNEEQSGDGSRHSGSRHHEASSRADSSRHSQVGQGQSSGPRTSRNQGSSVSQDSDSQGHSEDSERWSGSASRNHHGSAWEQSRDGSRHPGSHQEDRAGHGHSADSSRQSGTHHTESSSRGQAASSHEHARSSAGERHGSHHQQSADSSRHSGIGHGQASSAVRDSGHRGSSGSQASDSEGHSEDSDTQSLSAHGQAGPHQQSHQESTRGRSAERSGRSGSFLYQVSTHEQSESAHGRTGTSTGGRKRSLHEQARDSSRHSVSQEGQDTIHGHAGSSSGGRQGSHYEQLVDRSGHSGSHHSHTTSQGRSDAYHGQSGSRSASRQTHNDEQSGDGFRHSGSHHHEASSRADSSRHSQVGQGQSEGPRTSRHRESSVSQDSDSEGHSEDSERWSGSASRNHHGSAREQSRDGSRHPRSHHEDRAGHGHSADSSRQSGTRHTQTSSGGQAASSHEQGRSSAGERHGSRHQQSADSSRHSGIGHGQASSAVRDSGHRGYSGSQASDSEGHSEDSDTQSVSAHGQAGSHQQSHQESARGRSGERSGRSGSFLYQVSTHEQSESSHGWTGPSTRGRQGSRHEQAQDSSRHSASQEGQDTIRGHPGPSRGGRQGYHHEQSVDSSGHSGSHHSHITSQGSSHASHGQSGSRSASRTTRNDEQSVDGSRHSGSRHHEASTHADISRHSQAGQGQSEGSRTSRRQGSSVSQDSDSEGHSEDSERWSGSASRNHRGSAQEQSRDGSRHPRSHHEDRAGHGHSADSSRQSGTHHAETSSGGQAASSREQARSSPGERHGSRHQQSADSSRHSGIRRGQASSAVRDSGHWGSSGSQASDSEGHSEESDTQSVSGHGQDGPHQQSHQESARDRSGGRSGRSGSFLYQVSTHEQSESTHGQTGTSTGGRQGSHHEQARDSSRHSASQEGQDTIHAHPGSSRGGRQGSHHEQSVDTSGHSGSHHSHTTSQGRSDASHGQSGSRSASRQTRNDEQSGDGSRHSGSHHHEAFTQADSSRHSQSGQGESAGSRRSRRQGSSVSQDSDSQGHSEDSERWSGSASRNQHGSAREQSRDGSRHPGSHQEDRAGHGHSADSSRQSGTRHTESSSRGQAASSHEQARSSAGERHGSRHQLQSADSSRHSGIGHGQASSAVRDSGHRGSSGSQAIDSEGHSEDSDTQSVSAQGQAGPHQRSHKESARGQSGESSGRSGSFLYQVSTHEQPESTHGQSAPSTGGRQGSHHDQAQDSSRHSASQEGQDTIRGHPGPSRGGRQGSHHKQSVDRSGHSGSHHSHTTSQGRSDASRGQSGSRSASRQTHDKEQSGDGSRHSGSRHHEASSWADSSRHSQAGQGQSEGSRTSRRQGSSFSQDSDSEGHSEDSERRSGSASRNHRGSAQEQSRDGSRHPRSHHEDRAGHGHSADSSRQSGTHHAQNSSGGQAASFHEQARSSAGERHGSHHQQSADSSRHSGIGHGQASSAVRDSGHRGSSGSQASDSEGHSEDSDTQSVSAHGQAGPHQQSHQESTRGRSAERSGRSGSFLYQVSTHEQSESAHGRTGPSTGGRQGSRHEQARDSSRHSASQEGQDTIHGHPGSRRGGRQGSYHEQSVDRSGHSGSHHSHTTSQGRSDASHGQSGSRSASRQTRNEQQSGDGSRHSGSSHHEASTQADSSRHSQSGQGESAGSRRSRRQGSSVSQDSDSEAYPEDSERRSESASRNRHGSSREQSRDGSRHPGSSHRDTTRHVQSSPVQSDSSTAKEHGHFSSLSQDSAYRSGIQSRGSPHSSSSYHYQSEGTERQKGQSGLVWRHGSYGSADYDYGESGFRHSQHGSVSYNSNPVVFKERSDICKASAFGKDHPRYYATYINKDPGLCGHSSDISKQLGFSQSQRYYYYE

SEQ ID NO: 22 - protein, 819 aa, Bos taurus
MSTLLENINDIIKIFHKYSKTDKETDTLSEKELKELVEVEFRPILKNPGDPDTAEVFMYNLDRDHNNKIDFTEFFLMVFKVAQVYYSYTQRQNLQRAGQKQKKCTYHYGDEEDDTEEDKEETERKYSHSRSDGKTQDRSKSPRGRGKKRHGSKSGSKQRRGDTPTSGLRHGCSKKHESRREKKRRPSSTEPKERRHMSSVSPTRGYEEKEEEHGYENKGKTSAKCIGSEYDDSYQVCEDKVTTNFQPSHSKNYGSNITKGRDTEGHSRDTGRKSVFTHARSGSSSRNQNGSVQTHTGDNSTHSESQQETNSESVHRRSRNTGQRQGSHHEQSRDSSRHSGTRHGQPSTGSGGSRHRESSVSQASDSEGHSEDSGRQSVTTHGRPGSSSRNQHGSSQGQTGDSSRHSESHQGRHSDSLQRRSGTSTGQRQGSHHEQSRDSSRHSGTQQGQTSTGSGSSRHRDSSVSQASDSEGHSEDSGRQLETVLGIQSPIKEATVKLFMKGQDPARGKGRGATMSSQGTAPDTLELDMDKSQLNLEMADIGNPVLVKPVTVRDTPEIQVGILRQLMEDMVPAQGTNMDPPMVRQGTVIGTQRPIKGDTIRDTQEIQVGILRQLMEDLVPAQETNMDLPMARVETVLGTQSPIKRGIVNLSMEGQDPALGKDRGATMSSQETAPDTLELDMGKPQLNLEMADIGNPVLVKPVTVRDTPEIQVGILRQLMEGLVQAQETNMDLPRARQETVLGTQHIIKGGTVNLSMKGQRLELGKDRGATMSSQGTAPDSLELDMDIPLLDLEMADIGNPVLVSQVTERDTQEIQVDSP

SEQ ID NO: 23 - protein, 1183 aa, Macaca mulatta
MSTLLENIFAIINLFKQYSKKDKNTDTLSKKELKELLEKEFRPILKNPDDPDTVEVFMDHLDIDHNKKIDFTEFLLMVFKLAQAYYESTRKQNLPIAGHKHRKHSHHDKHEDNKEEENKEKRKRPLSLERRNNRKGNTGRSKSPRERGGKRHESSFEKKERKGYSPTYEEEEYGQNHHKSSKKEKNKTEITRLEHEGKRISERPEKKEEKEDGQFDYENAGRMDEKWTESGHIAIYHAIQDEVDDTTENILEENRRYETSRSPHDKSSLRVNRSPNANTSQIPLVEPRRRTRQRSSVSQDSDSEGHSEDSERQSESASRNHHGSVREQSRHGSRHPGSHHEDRAGHGHSADSSTQSGTRHTETSSRGQAVSSHEQARSSPGERHGSRHQQSAESSRHSGIGRGQASSAVSDRGHQGPSGSHFSDSEGHSEHSDTQSVSGHGQAGPHPHSHQESARGRSRERSGRSGSFLYQVSTHEQSESTHGRTGPSSAGRQGSRNEQARDSSRHSASHEVQDTVHGHHGSSRGRRQGSHHEQLVDSSGHSGSHHSHTTSQGRSDASRGESGARSASRQTRHEEQSGDGSRHSGSRHHESSNRADSSRHAQSSQGQSAGFRTSTRRGSSVSQDSDSEGHSEDSERQSESASRNHHGSVREQSRHGSGHSGSHHQDKVGHRYSGDSSRQSGTHHVETSSHGQAASSHEQTRSSPGERHGSHHQQSADSSRHSGTGRGQASSAVSDRGHQGPSGSHFSDSEGHSEHSDTQSVSGQGQAGRHPHSHQESARGRSGERSGRSGSFVYQVSTHEQSESTHGRTGPSTGGRQGSRNEQARDSSRHSVSHEGQDTIHGHHGSSRGGRQGSHHEQSVDSSGHSGSHHSHTTSQGRSDASRGESGARSASRQTRHEEQSGDGSRHSGSRHHEASNRADSSRHAQSGQGQSAGFRTSTRRGSSVSQDSDSEGHSEDSERQSESASRNHHGSVREQSRHGSRHPGSHHEDRAGHGHSADSSRQSGTRHTETSSRGQAVSSHEQARSSPGERHGSRHQQSAESSRHSGIGRGQASSAVSDRGHQGPSGSHFSDSEGHSEHSDTQSVSGHGQAGPHPHSHQESARGRSGERSGRSGSFLYQVSTHEQSESTHGRTGPSSAGRQGSRNEQARDSSRHSASHEVQDTVHGHHGSSRGGRQGSHHEQS

SEQ ID NO: 24 - protein, 2265 aa, Macaca mulatta
MTDLLRSVVTVIDVFYKYTKQDGECGTLSKDELKELLEKEFHPVLKNPDDPDTVDVIMHMLDRDHDRRLDFTEFLLMIFKLTMACNKVLSKEYCKASGSKKHRRGHRHQEEESETEEDEEDTPEHKSGYRHSSWSEGEEHGYSSGHSRGTVKRRHGSNSRRLGRQGHLSSSGNQERSQKRYHRSSSGHSWSSGKERHGFSSGELRERINKSHVSPSREFGEEYESGSGSKSWERKGHGGLSCGLEISGHESNSTQSRSSGQKLGSSRSCSGDSRRRSHACGYSNSSGCGRPQNASNSCQSHRFGGQVNQSSYIQSGCQSGINGEQGHDCVSGGQPSGCGQPESNSCSQSYSQRGYGARENGQPQNCGGQQRTGSSQSSFCGQYESGGSQSCSNGQHEHGSCGRFSNSSSSNEFSKCGKHRSGSGQFTSCEQHGTGLSQSSGFEQQVAGSSQTCSQYGSRSSQSSGYDEHGSSSGKTSGFGQHRSGSGHSSGFGQHGSGSGQSFGFGQHGSGSGQSSGFGQHESRSCQSSYGQHGSGSSQSSGYGQHASRQTSGFGQHGLGSGQSTGFGQYGSGSGQSSGFGQHGSGSGQSSGFGQHESRSGQSSYGQHSSSSSQSSGYGQHGSRQTSGFGQHGSGSGQSTGFGQYGSSLGQSSGFGQHGSGSGQSSGFGQHESTSGQSSYGQHGFGSSQSSGCGQHGLSSGQTSGFGQHELSSGQSSSFGQHGSGSGQSSGFRQHESGSGQSSGFGQHESRSHQSSYGPHGSGSGQSSGYGQHGSSSGQTSGFGQQGSSSSQYSGFGQHGSGLGQSSGFGQHGTGSGQFSGFGQHESRSHQSSYGQHGSGSSQSSGYGQHGSSSGHTTGFGQHRSSSGQYSGFGQHGSGLGQSSGFGQHGTGSGQSSGFGQHESRSHQSSYGQHGSGSSQSSSYGQHGSSSGQTSGFGQHRSGSGQSSGFGQYGLGSGQSSGFGQHGSGTGQSSGFARHEYRSGQSSYGQHGTGSSQSSGCGQRESGSGPTTGFGQHVSGSDNFSSSGQHISGSDQSTGFGQYGSGSGQSTGLGQVESQQVASGSTVHGRQETTHGQTINTARHSQSGQGQSTQTGSRVSRRRRSSQSENIDSEVHSRVSHRHSEHIDTQVGSHYPESGSTVHRRQGTTHGQRGDTTRHSHSGHGQSTQTGSRTTGRQRFSHSDATDSEVHSGVSHRPHSQEHTHGQDGSQLGESQSTVHERHETTYGQTGDATGHGYSGHGQSTQIGSRTSGRRGSGHSESSDTEVHSGGSHRPHSQEQTHGQARSQHGESRSTVHERHGTTHGQTGDTTRYAHYHNGQSAQRGSRTTGRGSGHSEYSDSELYSGGSHTYSGHTHGQAGSQHGESDSIVHERYGTTHGQTGDTTRHAHYSHGQSKQRGSRTTGRRGSGHSEYSDSEGHSGGSHTHSGHTHGHTHGQAGSQHGESGSSGHGGQGTTHGQTGDTTRHAHYGHGQSTQRGSRTAGRRGSGHSEYSDSEGHSGVSHTHSGHTHGQAGSQHGESESTVHERQQTTHGQTGDTTRHAHYGHGQSTQTGSRTTGRRGSGHSEYSDSEGHSGVSHTHSGHTHGQARSQHGESGSAIHGRQGTIHGQTGDTTRHGQSGHGQSTQTGSRTTGRRGSGHSEYSDSEGHSGGSHTHSGHTHGQAGSQHGESGSTVHGRQGTIHGQTGDTTRHGQSGHGQSIETGSRTTGRRGSGHSEYSDSEGHSGVSHTHSGHTHGQAGSQHGESESTVHERQQTTHGQTGDITEHGHSSHGQTTQTGSRTTGRRGSGHSEYSDSEWHSGGSHTHSGHTHGQAGFQHGESGSAVHGRQGTIHGQTGDTTRHGQSGHGESIQTGSRTIGRRGSGHSEYSDSEGHSGISHTHSGHTHGQAGSQHGESGSSGHGRQGTAHGQTGDTTRHAHYDHGQSTQRGSRTAGRRGSGHSEYSDSEGHSGVSHTHSGHTHGQAGSQHGESGAAVHGRQGIIHGQTGDTTRHGQSGQGQSTQRGSRTTGRRGSGHSEYSDSVGHSGVSHTHSGHTHGLAGSQHGESGSSGHGRQGTLHGQTGDTTRHAHYGHGQSTQRGSRTAGRRGSGHSEYSDSEWHSGGSHTHSGHTHGQAGSQHGESGSAVHGRQGTIHGQTGDTTRHGQSGHGQSTQIGPHSSSSYNYHSEGTERERGQSGLVWRHGSYGSADYDYGESRFRHSQHGSVSYNSNPVVFKERSDIRKASAFGEDHPRYYARYVNRQPGLYRHSSDISKQLGFSQSQRYYYYE

SEQ ID NO: 25 - protein, 12 aa, unidentified
  REGION 1..12: note = Description of Unknown: Human or Mouse filaggrin sequence
SGSQASDSEGHS

SEQ ID NO: 26 - protein, 24 aa, synthetic construct
  REGION 1..24: note = Description of Artificial Sequence: Synthetic peptide
  VARIANT 5: note = MOD_RES - Any amino acid
  VARIANT 10: note = MOD_RES - Any amino acid
  VARIANT 18: note = MOD_RES - Any amino acid
QSGEXSGRSXSFLYQVSXHEQSES

SEQ ID NO: 27 - protein, 345 aa, Homo sapiens
QSGESSGRSRSFLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLYQVSTHEQ

SEQ ID NO: 28 - protein, 345 aa, synthetic construct; identical to SEQ ID NO: 27
  REGION 1..345: note = Description of Artificial Sequence: Synthetic polypeptide

SEQ ID NO: 29 - protein, 7 aa, unidentified
  REGION 1..7: note = Description of Unknown: filaggrin sequence
QSGESSG

SEQ ID NO: 30 - DNA, 23 bp, synthetic construct
  misc_feature 1..23: note = Description of Artificial Sequence: Synthetic oligonucleotide
  source 1..23: mol_type = other DNA
gaagtgcggttcaacaccctccg

SEQ ID NO: 31 - DNA, 15 bp, unidentified
  misc_feature 1..15: note = Description of Unknown: operational taxonomic unit sequence
  source 1..15: mol_type = unassigned DNA
gatacagagatgcat

SEQ ID NO: 32 - DNA, 15 bp, unidentified
  misc_feature 1..15: note = Description of Unknown: operational taxonomic unit sequence
  source 1..15: mol_type = unassigned DNA
gatacagtgatgcat

SEQ ID NO: 33 - DNA, 15 bp, unidentified
  misc_feature 1..15: note = Description of Unknown: operational taxonomic unit sequence
  source 1..15: mol_type = unassigned DNA
gatacagtagatgca

SEQ ID NO: 34 - DNA, 15 bp, unidentified
  misc_feature 1..15: note = Description of Unknown: operational taxonomic unit sequence
  source 1..15: mol_type = unassigned DNA
taccagatttacata

SEQ ID NO: 35 - DNA, 14 bp, unidentified
  misc_feature 1..14: note = Description of Unknown: operational taxonomic unit sequence
  source 1..14: mol_type = unassigned DNA
taccagattacata

SEQ ID NO: 36 - DNA, 14 bp, unidentified
  misc_feature 1..14: note = Description of Unknown: operational taxonomic unit sequence
  source 1..14: mol_type = unassigned DNA
caggtaggataata

SEQ ID NO: 37 - DNA, 15 bp, unidentified
  misc_feature 1..15: note = Description of Unknown: operational taxonomic unit sequence
  source 1..15: mol_type = unassigned DNA
caggtaggataatta

SEQ ID NO: 38 - DNA, 15 bp, unidentified
  misc_feature 1..15: note = Description of Unknown: operational taxonomic unit sequence
  source 1..15: mol_type = unassigned DNA
caggtaggataaata

SEQ ID NO: 39 - DNA, 13 bp, unidentified
  misc_feature 1..13: note = Description of Unknown: operational taxonomic unit sequence
  source 1..13: mol_type = unassigned DNA
caggtaggaaata

SEQ ID NO: 40 - DNA, 14 bp, unidentified
  misc_feature 1..14: note = Description of Unknown: operational taxonomic unit sequence
  source 1..14: mol_type = unassigned DNA
gattacagattaca

SEQ ID NO: 41 - DNA, 15 bp, unidentified
  misc_feature 1..15: note = Description of Unknown: operational taxonomic unit sequence
  source 1..15: mol_type = unassigned DNA
gattacagatttaca

SEQ ID NO: 42 - DNA, 15 bp, unidentified
  misc_feature 1..15: note = Description of Unknown: operational taxonomic unit sequence
  source 1..15: mol_type = unassigned DNA
gattacagaattaca

SEQ ID NO: 43 - protein, 328 aa, Homo sapiens
SLYQVSTHEQPDSAHGRTGTSTGGRQGSHHEQARDSSRHSASQEGQDTIRGHPGSSRGGRQGSHHEQSVNRSGHSGSHHSHTTSQGRSDASHGQSGSRSASRQTRNEEQSGDGTRHSGSRHHEASSQADSSRHSQVGQGQSSGPRTSRNQGSSVSQDSDSQGHSEDSERWSGSASRNHHGSAQEQSRDGSRHPRSHHEDRAGHGHSADSSRKSGTRHTQNSSSGQAASSHEQARSSAGERHGSRHQLQSADSSRHSGTGHGQASSAVRDSGHRGSSGSQATDSEGHSEDSDTQSVSGHGQAGHHQQSHQESARDRSGERSRRSGSFLY

SEQ ID NO: 44 - protein, 327 aa, Homo sapiens
FLYQVSTHKQSESSHGWTGPSTGVRQGSHHEQARDNSRHSASQDGQDTIRGHPGSSRRGRQGSHHEQSVDRSGHSGSHHSHTTSQGRSDASRGQSGSRSASRTTRNEEQSRDGSRHSGSRHHEASSHADISRHSQAGQGQSEGSRTSRRQGSSVSQDSDSEGHSEDSERWSGSASRNHRGSAQEQSRHGSRHPRSHHEDRAGHGHSADSSRQSGTPHAETSSGGQAASSHEQARSSPGERHGSRHQQSADSSRHSGIPRRQASSAVRDSGHWGSSGSQASDSEGHSEESDTQSVSGHGQDGPHQQSHQESARDWSGGRSGRSGSFIY

SEQ ID NO: 45 - protein, 327 aa, Homo sapiens
FIYQVSTHEQSESAHGRTRTSTGRRQGSHHEQARDSSRHSASQEGQDTIRAHPGSRRGGRQGSHHEQSVDRSGHSGSHHSHTTSQGRSDASHGQSGSRSASRQTRKDKQSGDGSRHSGSRHHEAASWADSSRHSQVGQEQSSGSRTSRHQGSSVSQDSDSERHSDDSERLSGSASRNHHGSSREQSRDGSRHPGFHQEDRASHGHSADSSRQSGTHHTESSSHGQAVSSHEQARSSPGERHGSRHQQSADSSRHSGIGHRQASSAVRDSGHRGSSGSQVTNSEGHSEDSDTQSVSAHGQAGPHQQSHKESARGQSGESSGRSRSFLY

SEQ ID NO: 46 - protein, 327 aa, Homo sapiens
FLYQVSSHEQSESTHGQTAPSTGGRQGSRHEQARNSSRHSASQDGQDTIRGHPGSSRGGRQGSYHEQSVDRSGHSGYHHSHTTPQGRSDASHGQSGPRSASRQTRNEEQSGDGSRHSGSRHHEPSTRAGSSRHSQVGQGESAGSKTSRRQGSSVSQDRDSEGHSEDSERRSESASRNHYGSAREQSRHGSRNPRSHQEDRASHGHSAESSRQSGTRHAETSSGGQAASSQEQARSSPGERHGSRHQQSADSSTDSGTGRRQDSSVVGDSGNRGSSGSQASDSEGHSEESDTQSVSAHGQAGPHQQSHQESTRGQSGERSGRSGSFLY

SEQ ID NO: 47 - protein, 328 aa, Homo sapiens
FLYQVSTHEQSESAHGRTGPSTGGRQRSRHEQARDSSRHSASQEGQDTIRGHPGSSRGGRQGSHYEQSVDSSGHSGSHHSHTTSQERSDVSRGQSGSRSVSRQTRNEKQSGDGSRHSGSRHHEASSRADSSRHSQVGQGQSSGPRTSRNQGSSVSQDSDSQGHSEDSERWSGSASRNHLGSAWEQSRDGSRHPGSHHEDRAGHGHSADSSRQSGTRHTESSSRGQAASSHEQARSSAGERHGSHHQLQSADSSRHSGIGHGQASSAVRDSGHRGYSGSQASDSEGHSEDSDTQSVSAQGKAGPHQQSHKESARGQSGESSGRSGSFLY

SEQ ID NO: 48 - protein, 327 aa, Homo sapiens
FLYQVSTHEQSESTHGQSAPSTGGRQGSHYDQAQDSSRHSASQEGQDTIRGHPGPSRGGRQGSHQEQSVDRSGHSGSHHSHTTSQGRSDASRGQSGSRSASRKTYDKEQSGDGSRHSGSHHHEASSWADSSRHSLVGQGQSSGPRTSRPRGSSVSQDSDSEGHSEDSERRSGSASRNHHGSAQEQSRDGSRHPRSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLY

SEQ ID NO: 49 - protein, 327 aa, Homo sapiens
FLYQVSTHEQSESAHGRTGTSTGGRQGSHHKQARDSSRHSTSQEGQDTIHGHPGSSSGGRQGSHYEQLVDRSGHSGSHHSHTTSQGRSDASHGHSGSRSASRQTRNDEQSGDGSRHSGSRHHEASSRADSSGHSQVGQGQSEGPRTSRNWGSSFSQDSDSQGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLY

SEQ ID NO: 50 - protein, 327 aa, Homo sapiens
FLYQVSTHEQSESSHGWTGPSTRGRQGSRHEQAQDSSRHSASQDGQDTIRGHPGSSRGGRQGYHHEHSVDSSGHSGSHHSHTTSQGRSDASRGQSGSRSASRTTRNEEQSGDGSRHSGSRHHEASTHADISRHSQAVQGQSEGSRRSRRQGSSVSQDSDSEGHSEDSERWSGSASRNHHGSAQEQLRDGSRHPRSHQEDRAGHGHSADSSRQSGTRHTQTSSGGQAASSHEQARSSAGERHGSHHQQSADSSRHSGIGHGQASSAVRDSGHRGYSGSQASDNEGHSEDSDTQSVSAHGQAGSHQQSHQESARGRSGETSGHSGSFLY

SEQ ID NO: 51 - protein, 327 aa, Homo sapiens
FLYQVSTHEQSESSHGWTGPSTRGRQGSRHEQAQDSSRHSASQYGQDTIRGHPGSSRGGRQGYHHEHSVDSSGHSGSHHSHTTSQGRSDASRGQSGSRSASRTTRNEEQSGDSSRHSVSRHHEASTHADISRHSQAVQGQSEGSRRSRRQGSSVSQDSDSEGHSEDSERWSGSASRNHRGSVQEQSRHGSRHPRSHHEDRAGHGHSADRSRQSGTRHAETSSGGQAASSHEQARSSPGERHGSRHQQSADSSRHSGIPRGQASSAVRDSRHWGSSGSQASDSEGHSEESDTQSVSGHGQAGPHQQSHQESARDRSGGRSGRSGSFLY

SEQ ID NO: 52 - protein, 327 aa, Homo sapiens
FLYQVSTHEQSESAHGRTRTSTGRRQGSHHEQARDSSRHSASQEGQDTIRGHPGSSRRGRQGSHYEQSVDRSGHSGSHHSHTTSQGRSDASRGQSGSRSASRQTRNDEQSGDGSRHSWSHHHEASTQADSSRHSQSGQGQSAGPRTSRNQGSSVSQDSDSQGHSEDSERWSGSASRNHRGSAQEQSRDGSRHPTSHHEDRAGHGHSAESSRQSGTHHAENSSGGQAASSHEQARSSAGERHGSHHQQSADSSRHSGIGHGQASSAVRDSGHRGSSGSQASDSEGHSEDSDTQSVSAHGQAGPHQQSHQESTRGRSAGRSGRSGSFLY